(19) United States
(12) Patent Application Publication
(10) Pub. No.: US 2026/0091140 A1
(43) Pub. Date: Apr. 2, 2026

(54) GENE THERAPY FOR FOXG1 SYNDROME

(71) Applicant: THE BOARD OF REGENTS OF THE UNIVERSITY OF TEXAS SYSTEM, Austin, TX (US)

(72) Inventor: Sarah Elizabeth Sinnett, Coppell, TX (US)

(21) Appl. No.: 19/346,144

(22) Filed: Sep. 30, 2025

Related U.S. Application Data
(60) Provisional application No. 63/701,393, filed on Sep. 30, 2024.

(51) Int. Cl. A61K 48/00; A61K 38/17; A61P 25/08; C12N 15/86

(52) U.S. Cl. CPC A61K 48/0058; A61K 38/1709; A61P 25/08; C12N 15/86; C12N 2750/14143; C12N 2830/50

(57) ABSTRACT

Provided herein are expression constructs and viral vectors for use as gene therapies for FOXG1 haploinsufficiency. In various aspects, the expression constructs comprise a FOXG1 encoding nucleic acid and one or more miRNA regulatory elements that limit overexpression in vivo.

### CROSS-REFERENCE TO RELATED APPLICATIONS

This application claims priority to U.S. Provisional Application No. 63/701,393, filed Sep. 30, 2024, the contents of which are incorporated by reference herein in their entirety for all purposes.

### INCORPORATION BY REFERENCE OF SEQUENCE LISTING

The instant application contains a Sequence Listing which has been submitted electronically in XML format and is hereby incorporated by reference in its entirety for all purposes. The XML copy, created on Sep. 30, 2025, is referred to as UTSD.P3980US_SequenceListing.xml and is 33,668 bytes in size.

### FIELD

The present disclosure is directed to gene therapy compositions and methods for treating diseases associated with FOXG1 haploinsufficiency.

### BACKGROUND

Loss-of-function and duplication of the transcription regulator FOXG1 mediate FOXG1 haploinsufficiency syndrome and West syndrome respectively. The role of FOXG1 in patterning the embryonic telencephalon is well characterized, but relatively little is known about postnatal FOXG1 activity. FOXG1 haploinsufficiency syndrome is characterized by intellectual disability and seizures. West syndrome is also characterized by seizures. FOXG1 syndrome may therefore be a good candidate for a regulated gene therapy that permits efficacy without dose-dependent toxicity. The urgency for initiating a gene therapy program is strong, as patients have no FDA-approved genetic intervention. There is a need, therefore, for improved treatments for diseases and conditions characterized by dysregulated FOXG1 expression.

### SUMMARY

In various aspects, the present disclosure is directed to an expression construct comprising a transgene nucleic acid encoding FOXG1 and one or more miRNA regulatory elements.

In various aspects, the one or more miRNA regulatory elements comprise a micro-RNA responsive autoregulatory element (miRARE). In some aspects, the miRARE comprises a nucleic acid sequence having at least 70%, at least 75%, at least 80%, at least 85%, at least 90%, at least 95%, at least 96%, at least 97%, at least 98%, at least 99%, or 100% sequence identity to SEQ ID NO: 11.

In various aspects, the one or more miRNA regulatory elements comprise one or more miRNA binding sites. In various aspects, the one or more miRNA regulatory elements comprise one or more binding sites for a tissue specific miRNA. In various aspects, the one or more miRNA regulatory elements comprise a nucleic acid sequence having at least 70%, at least 75%, at least 80%, at least 85%, at least 90%, at least 95%, at least 96%, at least 97%, at least 98%, at least 99%, or 100% sequence identity to SEQ ID NO: 12.

In various aspects, the one or more miRNA regulatory elements comprise a nucleic acid sequence having at least 70%, at least 75%, at least 80%, at least 85%, at least 90%, at least 95%, at least 96%, at least 97%, at least 98%, at least 99%, or 100% sequence identity to SEQ ID NO: 11 and a nucleic acid sequence having at least 70%, at least 75%, at least 80%, at least 85%, at least 90%, at least 95%, at least 96%, at least 97%, at least 98%, at least 99%, or 100% sequence identity to SEQ ID NO: 12.

In various aspects, the one or more miRNA regulatory elements trigger miRNA directed degradation of a FOXG1 RNA transcript encoded by the expression construct. In various aspects, the miRNA directed degradation is tissue specific.

In various aspects, any of the expression constructs provided herein may comprise a viral vector. In various aspects, the viral vector comprises a recombinant adeno-associated viral vector (rAAV vector).

In various aspects, the rAAV vector comprises in a 5′ to 3′ direction a) a first AAV ITR sequence; b) a promoter sequence; c) a transgene nucleic acid molecule, wherein the transgene nucleic acid molecule comprises a nucleic acid sequence encoding for a FOXG1 polypeptide; d) a miRNA regulatory element, e) a polyA sequence; and f) a second AAV ITR sequence. In various aspects, the first AAV ITR sequence comprises SEQ ID NO: 1 or 2 and/or the second AAV ITR sequence comprises SEQ ID NO: 14 or 15. In various aspects, the promoter sequence comprises SEQ ID NO: 3. In various aspects, the miRNA regulatory element comprises SEQ ID NO: 11. In various aspects, the FOXG1 polypeptide comprises an amino acid sequence of SEQ ID NO: 5.

In various aspects, an AAV vector provided herein may comprise a nucleic acid sequence having at least 70%, at least 75%, at least 80%, at least 85%, at least 90%, at least 95%, at least 96%, at least 97%, at least 98%, at least 99%, or 100% sequence identity to SEQ ID NOs: 16 to 18.

Also provided are pharmaceutical composition comprising an expression construct (e.g., an rAAV vector) provided herein and at least one pharmaceutically appropriate carrier or excipient.

Also provided are methods of treating FOXG1 haploinsufficiency in a subject in need thereof, the method comprising administering a therapeutically effective amount of the pharmaceutical composition comprising an expression construct (e.g., an rAAV vector) to the subject. In various aspects, the subject has or is suspected of having West syndrome and the method further comprises treating West syndrome in the subject.

### BRIEF DESCRIPTION OF THE DRAWINGS

The above and further features will be more clearly appreciated from the following detailed description when taken in conjunction with the accompanying drawings. The patent or application file contains at least one drawing executed in color. Copies of this patent or patent application publication with color drawing(s) will be provided by the Office upon request and payment of the necessary fee.

FIG. 1 depicts diagrams of gene therapy vectors designed to evaluate neuronal expression and transduction efficiency (top) or to evaluate cytokine and miRNA profiles after maximum intraCSF dose (bottom).

FIG. 2 depicts diagrams of gene therapy vectors administered to 4 treatment groups (including one vehicle) in 4 brain regions (hippocampus, cortex, brain stem, cerebellum), and 1 spinal cord region (cervical). The promoter is the hSyn promoter. The “Targets” refer to four tandem targets for miR-122 (e.g., SEQ ID NO: 12).

FIG. 3 depicts representative immunofluorescence staining images of Pons of mice treated with indicated vectors and stained for NeuN (a neuronal marker, red), myc (green), and DAPI (a nuclei marker, blue).

FIGS. 4A-4B depict representative immunofluorescence (FIG. 4A) and quantification (FIG. 4B) of myc staining in the pons, midbraind, medulla, and cortex of treated mice.

FIG. 5 shows representative immunofluorescence staining showing myc expression (green) in cerebellum of mice treated with low (1E11 vg/mouse) or high (1E12 vg/mouse) of FOXG1-myc or FOXG1-myc-miRARE AAV vectors. NeuN stain (red) depicts neurons and DAPI stain (blue) depicts nuclei.

FIG. 6 shows bar graphs depicting IL-2, TNF-α and IFN-α levels in vehicle or vector treated mice.

FIG. 7 shows miRNA expression in hippocampus of mice treated with AAV9/hFOXG1, AAV9/hFOXG1-miRARE, or a vehicle vector (AAV9/EGFP) and shows that unregulated FOXG1 vector significantly changes expression of many miRNAs.

FIG. 8 depicts the number of up-regulated and down-regulated miRNAs observed after the treatments described in FIG. 7.

FIGS. 9A-9F show that untreated FOXG1cre/+ mice exhibit anxiety-related behavior in an open field test as measured by increased time in periphery (FIG. 9B), decreased time in center (FIG. 9C), decreased entries into center (FIG. 9D), and decreased percentage of time in center (FIG. 9E). Untreated mice showed no difference in distance moved relative to WT (FIG. 9A). FIG. 9F shows a time-lapse image of mouse movement during experiment.

FIGS. 10A-10D show untreated FOXG1cre/+ mice exhibit increased anxiety in a dark/light preference test as measured by increased latency to explore (FIG. 10A), decreased time in light chamber (FIG. 10B), increased time in dark chamber (FIG. 10C). FIG. 10D shows that untreated FOXG1cre/+shows and increased preference for a dark chamber relative to WT.

The drawing figures do not limit the present disclosure to the specific embodiments disclosed and described herein. The drawings are not necessarily to scale, emphasis instead being placed on clearly illustrating principles of certain embodiments of the present disclosure.

### DESCRIPTION

The following detailed description references the accompanying drawings that illustrate various embodiments of the present disclosure. The drawings and description are intended to describe aspects and embodiments of the present disclosure in sufficient detail to enable those skilled in the art to practice the present disclosure. Other components can be utilized and changes can be made without departing from the scope of the present disclosure. The following description is, therefore, not to be taken in a limiting sense.

The present disclosure is based, in part, on the surprising discovery that excessive and unregulated FOXG1 expression resulting from delivery of unregulated gene vectors to subjects with FOXG1 haploinsufficiency or otherwise dysregulated expression of FOXG1 can lead to intolerable side effects. To correct this, the inventors have designed a regulated FOXG1 vector that controls expression of FOXG1 to mitigate harmful effects of overexpression.

Accordingly, the present disclosure provides, inter alia, compositions and methods for treating a disease in a subject, the method comprising administering at least one therapeutically effective amount of a regulated recombinant adeno-associated virus (rAAV) viral vector encoding for FOXG1 and comprising at least one miRNA regulatory element. The present disclosure also provides, inter alia, isolated polynucleotides, recombinant adeno-associated virus (rAAV) vectors, and rAAV viral vectors comprising transgene nucleic acid molecules comprising nucleic acid sequences encoding for FOXG1 polypeptides. The present disclosure also provides methods of manufacturing these isolated polynucleotides, rAAV vectors, and rAAV viral vectors, as well as their use to deliver transgenes to treat or prevent a disease or disorder, including diseases associated with loss, misfunction and/or deficiency of a FOXG1 gene, including, but not limited to FOXG1 haploinsufficiency syndrome and West syndrome.

### I. Terminology

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present disclosure or the appended claims.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

The terms “comprising,” “including,” and “having” are used interchangeably in this disclosure. The terms “comprising,” “including” and “having” mean to include, but not necessarily be limited to the things so described.

The terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps, or acts are in some way inherently mutually exclusive.

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991), all of which are incorporated by reference herein. As used herein, the following terms have the meanings ascribed to them below, unless specified otherwise.

The phraseology and terminology employed herein are for the purpose of description and should not be regarded as limiting. When introducing elements of the present disclosure or the preferred aspects(s) thereof, the articles “a,” “an,” “the” and “said” are intended to mean that there are one or more of the elements. The terms “comprising,” “including” and “having” are intended to be inclusive and mean that there may be additional elements other than the listed elements. Wherever the terms “comprising” or “including” are used, it should be understood the disclosure also expressly contemplates and encompasses additional aspects “consisting of” the disclosed elements, in which additional elements other than the listed elements are not included.

The term “about” or “approximately,” as used herein, can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” can mean an acceptable error range for the particular value, such as 10% of the value modified by the term “about.” As used herein, the term “about,” can mean relative to the recited value, e.g., amount, dose, temperature, time, percentage, etc., ±10%, ±9%, ±8%, ±7%, ±6%, ±5%, ±4%, ±3%, ±2%, or ±1%.

Further, as the present disclosure is susceptible to aspects of many different forms, it is intended that the present disclosure be considered as an example of the principles of the present disclosure and not intended to limit the present disclosure to the specific aspects shown and described. Any one of the features of the present disclosure may be used separately or in combination with any other feature. References to the terms “aspect,” “aspects,” and/or the like in the description mean that the feature and/or features being referred to are included in, at least, one aspect of the description. Separate references to the terms “aspect,” “aspects,” and/or the like in the description do not necessarily refer to the same aspect and are also not mutually exclusive unless so stated and/or except as will be readily apparent to those skilled in the art from the description. For example, a feature, structure, process, step, action, or the like described in one aspect may also be included in other aspects but is not necessarily included. Thus, the present disclosure may include a variety of combinations and/or integrations of the aspects described herein. Additionally, all aspects of the present disclosure, as described herein, are not essential for its practice. Likewise, other systems, methods, features, and advantages of the present disclosure will be, or become, apparent to one with skill in the art upon examination of the figures and the description. It is intended that all such additional systems, methods, features, and advantages be included within this description, be within the scope of the present disclosure, and be encompassed by the claims.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues See, e.g., Batzer et al., Nucleic Acid Res. 19:5081 (1991), the disclosure of which is incorporated in its entirety herein.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

Within the context of the application a protein is represented by an amino acid sequence and correspondingly a nucleic acid molecule or a polynucleotide represented by a nucleic acid sequence. Identity and similarity between sequences: throughout this application, each time one refers to a specific amino acid sequence SEQ ID NO (take SEQ ID NO: Y as example), one may replace it by: a polypeptide represented by an amino acid sequence comprising a sequence that has at least 60% sequence identity or similarity with amino acid sequence SEQ ID NO: Y. Another preferred level of sequence identity or similarity is 65%. Another preferred level of sequence identity or similarity is 70%. Another preferred level of sequence identity or similarity is 75%. Another preferred level of sequence identity or similarity is 80%. Another preferred level of sequence identity or similarity is 85%. Another preferred level of sequence identity or similarity is 90%. Another preferred level of sequence identity or similarity is 95%. Another preferred level of sequence identity or similarity is 98%. Another preferred level of sequence identity or similarity is 99%.

Each amino acid sequence described herein by virtue of its identity or similarity percentage with a given amino acid sequence respectively has in a further preferred aspect an identity or a similarity of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% with the given nucleotide or amino acid sequence, respectively. The terms “homology,” “sequence identity” and the like are used interchangeably herein. Sequence identity is described herein as a relationship between two or more amino acid (polypeptide or protein) sequences or two or more nucleic acid (polynucleotide) sequences, as determined by comparing the sequences. In a preferred aspect, sequence identity is calculated based on the full length of two given SEQ ID NO's or on a part thereof. Part thereof preferably means at least 50%, 60%, 70%, 80%, 90%, or 100% of both SEQ ID NO's. In the art, “identity” also refers to the degree of sequence relatedness between amino acid or nucleic acid sequences, as the case may be, as determined by the match between strings of such sequences. The degree of sequence identity between two sequences can be determined, for example, by comparing the two sequences using computer programs commonly employed for this purpose, such as global or local alignment algorithms. Nonlimiting examples include BLASTp, BLASTn, Clustal W, MAFFT, Clustal Omega, AlignMe, Praline, GAP, BESTFIT, or another suitable method or algorithm. A Needleman and Wunsch global alignment algorithm can be used to align two sequences over their entire length or part thereof (part thereof may mean at least 50%, 60%, 70%, 80%, 90% of the length of the sequence), maximizing the number of matches and minimizes the number of gaps. Default settings can be used and preferred program is Needle for pairwise alignment (in an aspect, EMBOSS Needle 6.6.0.0, gap open penalty 10, gap extent penalty: 0.5, end gap penalty: false, end gap open penalty: 10, end gap extent penalty: 0.5 is used) and MAFFT for multiple sequence alignment (in an aspect, MAFFT v7Default value is: BLOSUM62 [bl62], Gap Open: 1.53, Gap extension: 0.123, Order: aligned, Tree rebuilding number: 2, Guide tree output: ON [true], Max iterate: 2, Perform FFTS: none is used).

“Similarity” between two amino acid sequences is determined by comparing the amino acid sequence and its conserved amino acid substitutes of one polypeptide to the sequence of a second polypeptide. Similar algorithms used for determination of sequence identity may be used for determination of sequence similarity. Optionally, in determining the degree of amino acid similarity, the skilled person may also take into account so-called conservative amino acid substitutions. As used herein, “conservative” amino acid substitutions refer to the interchangeability of residues having similar side chains.

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gin or His; Asp to Glu; Cys to Ser or Ala; Gin to Asn; Glu to Asp; Gly to Pro; His to Asn or Gin; lie to Leu or Vai; Leu to lie or Vai; Lys to Arg; Gin or Glu; Met to Leu or lie; Phe to Met, Leu or Tyr; Ser to Thr; Thr to Ser; Trp to Tyr; Tyr to Trp or Phe; and Vai to lie or Leu.

A “subject” of diagnosis or treatment is a cell or an animal such as a mammal, or a human. A subject is not limited to a specific species and includes non-human animals subject to diagnosis or treatment and those subject to infections or animal models, including, without limitation, simian, murine, rat, canine, or leporid species, as well as other livestock, sport animals, or pets. In some aspects, the subject is a human.

The term “administration” and variants thereof (e.g., “administering” a composition) in reference to a composition of the disclosure means introducing the composition or a prodrug of the composition into the system of the subject in need of treatment. When a composition of the disclosure or prodrug thereof is provided in combination with one or more other active agents (e.g., a cytotoxic agent, etc.), “administration” and its variants are each understood to include concurrent and sequential introduction of the composition or prodrug thereof and other agents. The present disclosure includes within its scope prodrugs of the compositions of this disclosure. In general, such prodrugs will be functional derivatives of the compositions of this disclosure which are readily convertible in vivo into the required composition. Thus, in the methods of treatment of the present disclosure, the term “administering” shall encompass the treatment of the various conditions described with the composition specifically disclosed or with a composition which may not be specifically disclosed, but which converts to the specified composition in vivo after administration to the patient.

The term “therapeutically effective amount” as used herein means that amount of active compound or pharmaceutical agent that elicits the biological or medicinal response in a tissue, system, animal, or human that is being sought by a researcher, veterinarian, medical doctor or other clinician.

As used herein, the term “treating” refers to the application or administration of a composition including one or more active agents to a subject, who is in need of the treatment, for example, having a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder. Alleviating a target disease/disorder includes delaying the development or progression of the disease or reducing disease severity. Alleviating the disease does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

### II. Compositions

Aspects of the present disclosure relate to expression constructs that allow for regulated expression of FOXG1 in a subject in need thereof. These expression constructs comprise isolated polynucleotides comprising a transgene nucleic acid molecule encoding for FOXG1 and at least one miRNA regulatory element. In various aspects, the expression constructs may comprise one or more promoters or regulatory elements operably linked to the transgene nucleic acid molecule. In various aspects, the expression constructs may be comprised in a viral vector (e.g., an recombinant AAV vector (rAAV). Each of these elements are described further herein below.

### Transgene Nucleic Acid Molecule

The present disclosure provides isolated polynucleotides comprising at least one transgene nucleic acid molecule.

In some aspects, a transgene nucleic acid molecule can comprise a nucleic acid sequence encoding a FOXG1 polypeptide, or at least one fragment thereof. In some aspects, a transgene nucleic acid molecule can comprise a nucleic acid sequence encoding a biological equivalent of a FOXG1 polypeptide.

FOXG1 (Forkhead Box G1) is a transcriptional regulator encoded by the FOXG1 gene, which plays a pivotal role in early brain development, particularly in the cerebral cortex. It governs key processes such as neural progenitor cell proliferation, neuronal migration, and cortical circuit assembly. FOXG1 is essential for proper cerebral patterning and the transition between neuronal layers during development, and its expression must be tightly regulated both spatially and temporally. Loss-of-function mutations in FOXG1 lead to FOXG1 syndrome, a severe neurodevelopmental disorder characterized by microcephaly, simplified gyral patterns, corpus callosum anomalies, intellectual disability, epilepsy, and motor and language deficits. This syndrome is often associated with haploinsufficiency, where one functional copy of the gene is insufficient for normal function. Additionally, FOXG1 dysfunction has been implicated in West syndrome, a form of infantile epileptic encephalopathy, and in autism spectrum disorder-like phenotypes due to disrupted GABAergic circuit formation.

In some aspects, a FOXG1 polypeptide comprises, consists essentially of, or consists of an amino acid sequence at least 65%, 70%, 75%, 80%, 85%, 90%, 95%, 96%, 97%, 98%, 99%, or 100% (or any percentage in between) identical to the amino acid sequence put forth in SEQ ID or a fragment thereof. In some aspects, a FOXG1 polypeptide comprises, consists essentially of, or consists of an amino acid sequence at least 65%, 70%, 75%, 80%, 85%, 90%, 95%, 96%, 97%, 98%, 99%, or 100% (or any percentage in between) identical to at least one portion of the amino acid sequence put forth in SEQ ID NO: 5, or a fragment thereof.

In some aspects, a nucleic acid sequence encoding a FOXG1 polypeptide comprises, consists essentially of, or consists of a nucleic acid sequence at least 65%, 70%, 75%, 80%, 85%, 90%, 95%, 96%, 97%, 98%, 99%, or 100% (or any percentage in between) identical to the nucleic acid sequence put forth SEQ ID NO: 20. In some aspects, a nucleic acid sequence encoding a FOXG1 polypeptide comprises, consists essentially of, or consists of a nucleic acid sequence at least 65%, 70%, 75%, 80%, 85%, 90%, 95%, 96%, 97%, 98%, 99%, or 100% (or any percentage in between) identical to the nucleic acid sequence put forth in SEQ ID NO: 20. In some instances, the nucleic acid sequence encoding a FOXG1 polypeptide further comprises a Kozak consensus sequence which is a nucleic acid motif that functions as the protein translation initiation site in most eukaryotic mRNA transcripts. An exemplary Kozak consensus sequence is gtcgaccgccacc (SEQ ID NO: 21). An exemplary nucleic acid sequence encoding for a FOXG1 polypeptide that further comprises a Kozak consensus sequence is provided herein as SEQ ID NO: 4.

In some aspects, the nucleic acid sequence encoding a FOXG1 polypeptide can be a codon optimized nucleic acid sequence that encodes for a FOXG1 polypeptide. A codon optimized nucleic acid sequence encoding a FOXG1 polypeptide can comprise, consist essentially of, or consist of a nucleic acid sequence that is no more than 65%, 70%, 75%, 80%, 85%, 90%, 95%, 96%, 97%, 98%, 99%, or any percentage in between identical to the wildtype human nucleic acid sequence encoding the FOXG1 polypeptide. As used herein, the “wildtype human nucleic acid sequence encoding the FOXG1 polypeptide” refers to the nucleic acid sequence that encodes the FOXG1 polypeptide in a human genome.

In some aspects, a codon optimized nucleic acid sequence encoding a FOXG1 polypeptide can comprise no donor splice sites. In some aspects, a codon optimized nucleic acid sequence encoding a FOXG1 polypeptide can comprise no more than about one, or about two, or about three, or about four, or about five, or about six, or about seven, or about eight, or about nine, or about ten donor splice sites. In some aspects, a codon optimized nucleic acid sequence encoding a FOXG1 polypeptide comprises at least one, or at least two, or at least three, or at least four, or at least five, or at least six, or at least seven, or at least eight, or at least nine, or at least ten fewer donor splice sites as compared to the wildtype human nucleic acid sequence encoding the FOXG1 polypeptide. Without wishing to be bound by theory, the removal of donor splice sites in the codon optimized nucleic acid sequence can unexpectedly and unpredictably increase expression of the FOXG1 polypeptide in vivo, as cryptic splicing is prevented. Moreover, cryptic splicing may vary between different subjects, meaning that the expression level of the FOXG1 polypeptide comprising donor splice sites may unpredictably vary between different subjects.

In some aspects, a codon optimized nucleic acid sequence encoding a FOXG1 polypeptide can have a GC content that differs from the GC content of the wildtype human nucleic acid sequence encoding the FOXG1 polypeptide. In some aspects, the GC content of a codon optimized nucleic acid sequence encoding a FOXG1 polypeptide is more evenly distributed across the entire nucleic acid sequence, as compared to the wildtype human nucleic acid sequence encoding the FOXG1 polypeptide. Without wishing to be bound by theory, by more evenly distributing the GC content across the entire nucleic acid sequence, the codon optimized nucleic acid sequence exhibits a more uniform melting temperature (Tm) across the length of the transcript. The uniformity of melting temperature results unexpectedly in increased expression of the codon optimized nucleic acid in a human subject, as transcription and/or translation of the nucleic acid sequence occurs with less stalling of the polymerase and/or ribosome.

In some aspects, the codon optimized nucleic acid sequence encoding a FOXG1 polypeptide exhibits at least 5%, at least 10%, at least 20%, at least 30%, at least 50%, at least 75%, at least 100%, at least 200%, at least 300%, at least 500%, or at least 1000% increased expression in a human subject relative to a wild type or non-codon optimized nucleic acid sequence encoding an FOXG1 polypeptide.

In some aspects, a FOXG1 polypeptide can further comprise a protein tag. Without wishing to be bound by theory, the inclusion of a protein tag can allow for the detection and/or visualization of the exogenous FOXG1 polypeptide. As would be appreciated by the skilled artisan, non-limiting examples of protein tags include Myc tags, poly-histidine tags, FLAG-tags, HA-tags, SBP-tags or any other protein tag known in the art. In some aspects, the protein tag may be linked to the FOXG1 polypeptide via a flexible linker. Suitable linkers are known in the art and can include, for example, GSSGSSG (SEQ ID NO: 9). An exemplary nucleic acid encoding a myc peptide is provided herein as SEQ ID NO: 6. An exemplary nucleic acid encoding a myc peptide and a flexible linker (GSSGSSG) is provided herein as SEQ ID NO: 8.

Transgene nucleic acid molecules can comprise, consist essentially of, or consist of any of the transgene nucleic acid molecules described herein above.

miRNA Regulatory Elements

The present disclosure provides isolated polynucleotides comprising one or more miRNA regulatory elements.

The one or more miRNA regulatory elements can, in some aspects, comprise one or more binding sites for an miRNA (e.g., a tissue specific miRNA such as miR-122). In this way, transcripts encoded by the expression constructs may be selectively targeted and degraded by certain miRNAs, limiting, for instance, expression in certain organs or tissues (e.g., hepatic tissue). Alternatively, or in addition, the miRNA regulatory element can in some aspects comprise a micro-RNA responsive autoregulatory element, which is an element that triggers miRNA targeted degradation of the transcript at high levels of expression. An exemplary micro-RNA responsive autoregulatory element is provided herein as miRARE, as described in Sinnett S E, et al Brain 2021; 144 (10): 3005-3019 and Sadhu C, et al., Genes (Basel) 2023; 15 (1), which are incorporated by reference herein in their entirety).

In some aspects, the miRNA regulatory element comprises a miRARE nucleic acid sequence having at least 70%, at least 75%, at least 80%, at least 85%, at least 90%, at least 95%, at least 96%, at least 97%, at least 98%, at least 99%, or 100% sequence identity to SEQ ID NO: 11, provided herein below. In some aspects, the miRNA regulatory element comprises SEQ ID NO: 11.

In some aspects, the miRNA regulatory element comprises a regulatory binding site panel comprising one or more (e.g., two or more, three or more, or four or more) binding sites for an miRNA (e.g., miR-122). In some aspects, the miRNA regulatory element comprises a nucleic acid having at least 70%, at least 75%, at least 80%, at least 85%, at least 90%, at least 95%, at least 96%, at least 97%, at least 98%, at least 99%, or 100% sequence identity to SEQ ID NO: 12, provided herein below. In some aspects, the miRNA regulatory element comprises or consists of SEQ ID NO: 12.

In various aspects, the polynucleotides may comprise more than one miRNA regulatory element provided herein. For example, in some aspects, the polynucleotide may comprise a miRARE nucleic acid sequence (e.g., SEQ ID NO: 11) and a regulatory binding site panel (e.g., SEQ ID NO: 12). When used herein, the term miRNA regulatory element may refer to one or more miRNA regulatory elements provided herein and is inclusive of any and all combinations (e.g., in any order) of the miRNA regulatory elements provided herein. In some aspects, the miRNA regulatory element may exclude one of the miRNA regulatory elements herein (e.g., may not comprise SEQ ID NO: 11 or SEQ ID NO: 12). In preferred aspects, the miRNA regulatory element comprises the miRARE regulatory element comprising SEQ ID NO: 11 or a nucleic acid having at least 70%, at least 75%, at least 80%, at least 85%, at least 90%, at least 95%, at least 96%, at least 97%, at least 98%, at least 99%, or 100% sequence identity to SEQ ID NO: 11.

In any of these aspects, the miRNA regulatory element may trigger miRNA directed degradation of a FOXG1 RNA transcript encoded by the expression construct, optionally, wherein the miRNA directed degradation is tissue specific.

In certain embodiments, the miRNA regulatory element reduces expression of a transgene by at least about 1% to 75% relative to a corresponding nucleic acid molecule that does not comprise the miRNA regulatory element. In various embodiments, the reduction in transgene expression may be at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, or 75%. In further embodiments, the reduction may fall within a range selected from about 1% to 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, and about 70% to 75%.

### Regulatory Elements

The present disclosure provides isolated polynucleotides comprising at least one regulatory element operably linked to a transgene nucleic acid as provided above. In some aspects, the regulatory element comprises a promoter and/or an enhancer.

The term “promoter” and “promoter sequence” as used herein means a control sequence that is a region of a polynucleotide sequence at which the initiation and rate of transcription of a coding sequence, such as a gene or a transgene, are controlled. Promoters may be constitutive, inducible, repressible, or tissue-specific, for example. Promoters may contain genetic elements at which regulatory proteins and molecules such as RNA polymerase and transcription factors may bind. Non-limiting exemplary promoters include Rous sarcoma virus (RSV) LTR promoter (optionally with the RSV enhancer), a cytomegalovirus (CMV) promoter, an SV40 promoter, a dihydrofolate reductase promoter, a b-actin promoter, a phosphoglycerol kinase (PGK) promoter, a U6 promoter, a synapsin promoter, an HI promoter, a ubiquitous chicken b-actin hybrid (CBh) promoter, a small nuclear RNA (Ula or Ulb) promoter, an MECP2 promoter, an MeP418 promoter, an MeP426 promoter, a human variant of the MeP426 promoter, a minimal MECP2 promoter, a VMD2 promoter, an mRho promoter, or an EF1 promoter.

Additional non-limiting exemplary promoters provided herein include, but are not limited to EFla, Ubc, human b-actin, CAG, TRE, Ac5, Polyhedrin, CaMKIIa, Gall, TEF1, GDS, ADH1, Ubi, and a-1-antitrypsin (hAAT). It is known in the art that the nucleotide sequences of such promoters may be modified in order to increase or decrease the efficiency of mRNA transcription. See, e.g., Gao et al. (2018) Mol. Ther.: Nucleic Acids 12:135-145 (modifying TATA box of 7SK, U6 and HI promoters to abolish RNA polymerase III transcription and stimulate RNA polymerase II-dependent mRNA transcription). Synthetically-derived promoters may be used for ubiquitous or tissue specific expression. Further, virus-derived promoters, some of which are noted above, may be useful in the methods disclosed herein, e.g., CMV, HIV, adenovirus, and AAV promoters. In some aspects, the promoter is used together with at least one enhancer to increase the transcription efficiency. Non-limiting examples of enhancers include an interstitial retinoid-binding protein (IRBP) enhancer, an RSV enhancer or a CMV enhancer.

In some aspects, a promoter sequence can comprise, consist essentially of, or consist of a Rous sarcoma virus (RSV) LTR promoter sequence (optionally with the RSV enhancer), a cytomegalovirus (CMV) promoter sequence, an SV40 promoter sequence, a dihydrofolate reductase promoter sequence, a JeT promoter sequence, a strong a b-actin promoter sequence, a phosphoglycerol kinase (PGK) promoter sequence, a U6 promoter sequence, synapsin promoter, an HI promoter sequence, a ubiquitous chicken b-actin hybrid (CBh) promoter sequence, a small nuclear RNA (Ula or Ulb) promoter sequence, an MECP2 promoter sequence, an MeP418 promoter, an MeP426 promoter sequence, a small ubiquitous promoter sequence (also known as a Jetl promoter sequence) MECP2 promoter sequence, a VMD2 promoter sequence, an mRho promoter sequence, an EFI promoter sequence, an EFla promoter sequence, a Ubc promoter sequence, a human b-actin promoter sequence, a CAG promoter sequence, a TRE promoter sequence, an Ac5 promoter sequence, a Polyhedrin promoter sequence, a CaMKIIa promoter sequence, a Gall promoter sequence, a TEF1 promoter sequence, a GDS promoter sequence, an ADH1 promoter sequence, a Ubi promoter sequence, a MeP426 promoter, or an a-1-antitrypsin (hAAT) promoter sequence.

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) or synthetic techniques such that transcription of that gene is directed by the linked enhancer/promoter. Non-limiting examples of linked enhancer/promoter for use in the methods, compositions and constructs provided herein include a PDE promoter plus IRBP enhancer or a CMV enhancer plus Ula promoter. It is understood in the art that enhancers can operate from a distance and irrespective of their orientation relative to the location of an endogenous or heterologous promoter. It is thus further understood that an enhancer operating at a distance from a promoter is thus “operably linked” to that promoter irrespective of its location in the vector or its orientation relative to the location of the promoter.

As used throughout the disclosure, the term “operably linked” refers to the expression of a gene (i.e. a transgene) that is under the control of a promoter with which it is spatially connected. A promoter can be positioned 5′ (upstream) or 3′ (downstream) of a gene under its control. A promoter can be positioned 5′ (upstream) of a gene under its control. The distance between a promoter and a gene can be approximately the same as the distance between that promoter and the gene it controls in the gene from which the promoter is derived. Variation in the distance between a promoter and a gene can be accommodated without loss of promoter function.

In some aspects, a promoter sequence can comprise, consist essentially of, or consist of an alpha-synuclein promoter sequence. An alpha-synuclein promoter sequence can comprise, consist essentially of, or consist of a nucleic acid sequence at least 65%, 70%, 75%, 80%, 85%, 90%, 95%, 96%, 97%, 98%, 99%, or 100% (or any percentage in between) identical to the nucleic acid sequence put forth in SEQ ID NO: 3.

In some aspects, the isolated nucleic acids comprise a polvA sequence. In some aspects, apolyadenylation (poly A) sequence can comprise any polyA sequence known in the art. Non-limiting examples of polyA sequences include, but are not limited to, an MECP2 polyA sequence, a retinol dehydrogenase 1 (RDH1) polyA sequence, a bovine growth hormone (BGH) polyA sequence, an SV40 polyA sequence, a SPA49 polyA sequence, a SNRP-TK65 polyA sequence, a sNRP polyA sequence, or a TK65 polyA sequence.

Thus, a polyA sequence can comprise, consist essentially of, or consist of an MeCP2 polyA sequence, a retinol dehydrogenase 1 (RDH1) polyA sequence, a bovine growth hormone (BGH) polyA sequence, an SV40 polyA sequence, a SPA49 polyA sequence, a sNRP-TK65 polyA sequence, a sNRP polyA sequence, or a TK65 polyA sequence.

In some aspects, a polyA sequence can comprise, consist essentially of, or consist of a nucleic acid sequence at least 65%, 70%, 75%, 80%, 85%, 90%, 95%, 96%, 97%, 98%, 99%, or 100% (or any percentage in between) identical the sequence put forth in SEQ ID NO: 13.

### Vectors

In some aspects, the isolated polynucleotides comprising at least one transgene nucleic acid molecule described herein can be a vector. In some aspects, the isolated polynucleotides comprising at least one transgene nucleic acid molecule described herein can be an adeno-associated viral vector (AAV), and in some aspects can be a recombinant AAV (rAAV) vector.

As used herein, the term “vector” refers to a nucleic acid comprising, consisting essentially of, or consisting of an intact replicon such that the vector may be replicated when placed within a cell, for example by a process of transfection, infection, or transformation. It is understood in the art that once inside a cell, a vector may replicate as an extrachromosomal (episomal) element or may be integrated into a host cell chromosome. Vectors may include nucleic acids derived from retroviruses, adenoviruses, herpesvirus, baculoviruses, modified baculoviruses, papovaviruses, or otherwise modified naturally occurring viruses. Exemplary non-viral vectors for delivering nucleic acid include naked DNA; DNA complexed with cationic lipids, alone or in combination with cationic polymers; anionic and cationic liposomes; DNA-protein complexes and particles comprising, consisting essentially of, or consisting of DNA condensed with cationic polymers such as heterogeneous polylysine, defined-length oligopeptides, and polyethyleneimine, in some cases contained in liposomes; and the use of ternary complexes comprising, consisting essentially of, or consisting of a virus and polylysine-DNA.

A “viral vector” is defined as a recombinantly produced virus or viral particle that contains a polynucleotide to be delivered into a host cell, either in vivo, ex vivo or in vitro. Examples of viral vectors include retroviral vectors, AAV vectors, lentiviral vectors, adenovirus vectors, alphavirus vectors and the like. Alphavirus vectors, such as Semliki Forest virus-based vectors and Sindbis virus-based vectors, have also been developed for use in gene therapy and immunotherapy. See, e.g., Schlesinger and Dubensky (1999) Curr. Opin. Biotechnol. 5:434-439 and Ying, et al. (1999) Nat. Med. 5 (7): 823-827.

With respect to general recombinant techniques, vectors that contain both a promoter and a cloning site into which a polynucleotide can be operatively linked are well known in the art. Such vectors are capable of transcribing RNA in vitro or in vivo, and are commercially available from sources such as Agilent Technologies (Santa Clara, Cali!) and Promega Biotech (Madison, Wis.). In order to optimize expression and/or in vitro transcription, it may be necessary to remove, add or alter 5′ and/or 3′ untranslated portions of cloned transgenes to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

An “rAAV vector” as used herein refers to a vector comprising, consisting essentially of, or consisting of one or more transgene nucleic acid molecules and one or more AAV inverted terminal repeat sequences (ITRs). Such AAV vectors can be replicated and packaged into infectious viral particles when present in a host cell that provides the functionality of rep and cap gene products; for example, by transfection of the host cell. In some aspects, AAV vectors contain a promoter, at least one nucleic acid that may encode at least one protein or RNA, and/or an enhancer and/or a terminator within the flanking ITRs that is packaged into the infectious AAV particle. The encapsidated nucleic acid portion may be referred to as the AAV vector genome. Plasmids containing rAAV vectors may also contain elements for manufacturing purposes, e.g., antibiotic resistance genes, origin of replication sequences etc., but these are not encapsidated and thus do not form part of the AAV particle.

In some aspects, an rAAV vector can comprise at least one transgene nucleic acid molecule. In some aspects, an rAAV vector can comprise at least one AAV inverted terminal (ITR) sequence. In some aspects, an rAAV vector can comprise at least one promoter sequence. In some aspects, an rAAV vector can comprise at least one enhancer sequence. In some aspects, an rAAV vector can comprise at least one polyA sequence. In some aspects, an rAAV vector can comprise a RepCap sequence, which is a combined replication (Rep) and capsid (Cap) gene useful for capsid optimization to improve delivery efficiency, reduce immune response and enhance specificity to target tissues such as CNS.

In some aspects, an rAAV vector can comprise a first AAV ITR sequence, a promoter sequence, a transgene nucleic acid molecule, a miRNA regulatory element, and a second AAV ITR sequence. In some aspects, an rAAV vector can comprise, in the 5′ to 3′ direction, a first AAV ITR sequence, a promoter sequence, a transgene nucleic acid molecule, an miRNA regulatory element, and a second AAV ITR sequence.

In some aspects, an rAAV vector can comprise a first AAV ITR sequence, a promoter sequence, a transgene nucleic acid molecule, a miRNA regulatory element, a polyA sequence, and a second AAV ITR sequence. In some aspects, an rAAV vector can comprise, in the 5′ to 3′ direction, a first AAV ITR sequence, a promoter sequence, a transgene nucleic acid molecule, an miRNA regulatory element, a polyA sequence, and a second AAV ITR sequence.

In some aspects, an rAAV vector can comprise more than one transgene nucleic acid molecule. In some aspects, an rAAV vector can comprise at least two transgene nucleic acid molecules, such that the rAAV vector comprises a first transgene nucleic acid molecule and an at least second transgene nucleic acid molecule. In some aspects, the first and the at least second transgene nucleic acid molecule can comprise the same nucleic acid sequence. In some aspects, the first and the at least second transgene nucleic acid molecules can comprise different nucleic acid sequences. In some aspects, the first and at least second transgene nucleic acid sequences can be adjacent to each other.

In some aspects, an rAAV vector can comprise more than one promoter sequence. In some aspects, an rAAV vector can comprise at least two promoter sequences, such that the rAAV vector comprises a first promoter sequence and an at least second promoter sequence. In some aspects, the first and the at least second promoter sequences can comprise the same sequence. In some aspects, the first and the at least second promoter sequences can comprise different sequences. In some aspects, the first and the at least second promoter sequences can be adjacent to each other. In some aspects wherein an rAAV vector also comprises a first transgene nucleic acid molecule and an at least second transgene nucleic acid molecule, the first promoter can be located upstream (5′) of the first transgene nucleic acid molecule and the at least second promoter can be located between the first transgene nucleic acid molecule and the at least second transgene nucleic acid molecule, such that the at least second promoter is downstream (3′) of the first transgene nucleic acid molecule and upstream (5′) of the at least second transgene nucleic acid molecule.

Any of the preceding rAAV vectors can further comprise at least one enhancer. The at least one enhancer can be located anywhere in the rAAV vector. In some aspects, the at least one enhancer can be located immediately upstream (5′) of a promoter. Thus, an rAAV vector can comprise, in the 5′ to 3′ direction, a first AAV ITR sequence, an enhancer, a promoter sequence, a transgene nucleic acid molecule, a polyA sequence, a miRNA regulatory element, and a second AAV ITR sequence. In some aspects, the at least one enhancer can be located immediately downstream (3′) of a promoter. Thus, an rAAV vector can comprise, in the 5′ to 3′ direction, a first AAV ITR sequence, a promoter sequence, an enhancer, a transgene nucleic acid molecule, a polyA sequence, an miRNA regulatory element, and a second AAV ITR sequence. In some aspects, the at least one enhancer can be located immediately downstream of a transgene nucleic acid molecule. Thus, an rAAV vector can comprise, in the 5′ to 3′ direction, a first AAV ITR sequence, a promoter sequence, a transgene nucleic acid molecule, an enhancer, a polyA sequence, an miRNA regulatory element, and a second AAV ITR sequence.

### AAV ITR Sequences

In some aspects, an AAV ITR sequence can comprise any AAV ITR sequence known in the art. In some aspects, an AAV ITR sequence can be an AAV1 ITR sequence, an AAV2 ITR sequence, an AAV4 ITR sequence, an AAV5 ITR sequence, an AAV6 ITR sequence, an AAV7 ITR sequence, an AAV8 ITR sequence, an AAV9 ITR sequence, an AAV 10ITR sequence, an AAV11 ITR sequence, an AAV12 ITR sequence, an AAV13 ITR sequence, an AAVrh74 ITR sequence or an AAVrh. 10ITR sequence.

Thus, in some aspects, an AAV ITR sequence can comprise, consist essentially of, or consist of an AAV1 ITR sequence, an AAV2 ITR sequence, an AAV4 ITR sequence, an AAV5 ITR sequence, an AAV6 ITR sequence, an AAV7 ITR sequence, an AAV8 ITR sequence, an AAV9 ITR sequence, an AAV 10ITR sequence, an AAV 11 ITR sequence, an AAV 12 ITR sequence, an AAV13 ITR sequence, an AAVrh74 ITR sequence, or an AAVrh. 10ITR sequence. In some aspects, an AAV ITR sequence can comprise an AAV9 ITR sequence.

In some aspects, rAAV vector comprises a first AAV9 ITR sequence comprising a nucleic acid sequence having at least 70%, at least 75%, at least 80%, at least 85%, at least 90%, at least 95%, at least 96%, at least 97%, at least 98%, at least 99%, or 100% sequence identity to SEQ ID NO: 1 or 2. In some aspects, the rAAV vector comprises a second AAV9 ITR sequence comprising a nucleic acid sequence having at least 70%, at least 75%, at least 80%, at least 85%, at least 90%, at least 95%, at least 96%, at least 97%, at least 98%, at least 99%, or 100% sequence identity to SEQ ID NO: 14 or 15. In some aspects, an rAAV vector provided herein comprises a first AAV9 ITR sequence comprising SEQ ID NO: 1 and/or a second AAV9 ITR sequence comprising SEQ ID NO: 14. In some aspects, an rAAV vector provided herein comprises a first AAV9 ITR sequence comprising SEQ ID NO: 1 and/or a second AAV9 ITR sequence comprising SEQ ID NO: 15. In some aspects, an rAAV vector provided herein comprises a first AAV9 ITR sequence comprising SEQ ID NO: 2 and/or a second AAV9 ITR sequence comprising SEQ ID NO: 14. In some aspects, an rAAV vector provided herein comprises a first AAV9 ITR sequence comprising SEQ ID NO: 2 and/or a second AAV9 ITR sequence comprising SEQ ID NO: 15.

### Bacterial Plasmids

In some aspects, the rAAV vectors of the present disclosure can be contained within a bacterial plasmid to allow for propagation of the rAAV vector in vitro. Thus, the present disclosure provides bacterial plasmids comprising any of the rAAV vectors described herein. A bacterial plasmid can further comprise an origin of replication sequence. A bacterial plasmid can further comprise an antibiotic resistance gene. A bacterial plasmid can further comprise a resistance gene promoter.

### Origin of Replication Sequence

In some aspects, an origin of replication sequence can comprise, consist essentially of, or consist of any origin of replication sequence known in the art. The origin of replication sequence can be a bacterial origin of replication sequence, thereby allowing the rAAV vector comprising said bacterial origin of replication sequence to be produced, propagated and maintained in bacteria, using methods standard in the art.

### Antibiotic Resistance Genes

In some aspects, bacterial plasmids, rAAV vectors and/or rAAV viral vectors of the disclosure can comprise an antibiotic resistance gene.

In some aspects, an antibiotic resistance gene can comprise, consist essentially of, or consist of any antibiotic resistance genes known in the art. Examples of antibiotic resistance genes known in the art include, but are not limited to kanamycin resistance genes, spectinomycin resistance genes, streptomycin resistance genes, ampicillin resistance genes, carbenicillin resistance genes, bleomycin resistance genes, erythromycin resistance genes, polymyxin B resistance genes, tetracycline resistance genes and chloramphenicol resistance genes.

Exemplary rAAV Vectors

In some aspects, an rAAV vector of the present disclosure can comprise, consist essentially of, or consist of a nucleic acid sequence at least 65%, 70%, 75%, 80%, 85%, 90%, 95%, 96%, 97%, 98%, 99%, or 100% (or any percentage in between) identical to the sequence put forth in any one of SEQ ID NOs: 16 to 19. In some aspects, an rAAV vector of the present disclosure can comprise, consist essentially of, or consist of a nucleic acid sequence at least 65%, 70%, 75%, 80%, 85%, 90%, 95%, 96%, 97%, 98%, 99%, or 100% (or any percentage in between) identical to the sequence put forth in SEQ ID NO: 18. In some aspects, an rAAV vector of the present disclosure can comprise, consist essentially of, or consist of a nucleic acid sequence as put forth in SEQ ID NO: 18.

### AAV Capsids

An “AAV virion” or “AAV viral particle” or “AAV viral vector” or “rAAV viral vector” or “AAV vector particle” or “AAV particle” refers to a viral particle composed of at least one AAV capsid protein and an encapsidated polynucleotide rAAV vector. Thus, production of an rAAV viral vector necessarily includes production of an rAAV vector, as such a vector is contained within an rAAV vector.

As used herein, the term “viral capsid” or “capsid” refers to the proteinaceous shell or coat of a viral particle. Capsids function to encapsidate, protect, transport, and release into the host cell a viral genome. Capsids are generally comprised of oligomeric structural subunits of protein (“capsid proteins”). As used herein, the term “encapsidated” means enclosed within a viral capsid. The viral capsid of AAV is composed of a mixture of three viral capsid proteins: VP1, VP2, and VP3. The mixture of VP1, VP2 and VP3 contains 60 monomers that are arranged in a T=1 icosahedral symmetry in a ratio of 1:1:10 (VP1:VP2:VP3) or 1:1:20 (VP1:VP2:VP3) as described in Sonntag F et al., (June 2010). “A viral assembly factor promotes AAV2 capsid formation in the nucleolus”. Proceedings of the National Academy of Sciences of the United States of America. 107 (22): 10220-5, and Rabinowitz J E, Samulski R J (December 2000). “Building a better vector: the manipulation of AAV virions”. Virology. 278 (2): 301-8, each of which is incorporated herein by reference in its entirety.

The present disclosure provides an rAAV viral vector comprising: a) any of the rAAV vectors described herein, or complement thereof; and b) an AAV capsid protein.

The present disclosure provides an rAAV viral vector comprising: a) any of the rAAV vectors described herein; and b) an AAV capsid protein.

An AAV capsid protein can be any AAV capsid protein known in the art. An AAV capsid protein can be an AAV1 capsid protein, an AAV2 capsid protein, an AAV4 capsid protein, an AAV5 capsid protein, an AAV6 capsid protein, an AAV7 capsid protein, an AAV 8 capsid protein, an AAV9 capsid protein, an AAV 10capsid protein, an AAV 11 capsid protein, an AAV12 capsid protein, an AAV13 capsid protein, an AAVPHP.B capsid protein, an AAVrh74 capsid protein or an AAVrh. 10capsid protein. In some aspects, an AAV capsid protein comprises an AAV9 capsid protein.

### Compositions and Pharmaceutical Compositions

The present disclosure provides compositions comprising any of the isolated polynucleotides, rAAV vectors, and/or rAAV viral vectors described herein. In some aspects, the compositions can be pharmaceutical compositions. Accordingly, the present disclosure provides pharmaceutical compositions comprising any of the isolated polynucleotides, rAAV vectors, and/or rAAV viral vectors described herein.

The pharmaceutical composition, as described herein, may be formulated by any methods known or developed in the art of pharmacology, which include but are not limited to contacting the active ingredients (e.g., viral particles or recombinant vectors) with an excipient and/or additive and/or other accessory ingredient, dividing or packaging the product to a dose unit. The viral particles of this disclosure may be formulated with desirable features, e.g., increased stability, increased cell transfection, sustained or delayed release, biodistributions or tropisms, modulated or enhanced translation of encoded protein in vivo, and the release profile of encoded protein in vivo.

As such, the pharmaceutical composition may further comprise saline, lipidoids, liposomes, lipid nanoparticles, polymers, lipoplexes, core-shell nanoparticles, peptides, proteins, cells transfected with viral vectors (e.g., for transplantation into a subject), nanoparticle mimics or combinations thereof. In some aspects, the pharmaceutical composition is formulated as a nanoparticle. In some aspects, the nanoparticle is a self-assembled nucleic acid nanoparticle.

A pharmaceutical composition in accordance with the present disclosure may be prepared, packaged, and/or sold in bulk, as a single unit dose, and/or as a plurality of single unit doses. The amount of the active ingredient is generally equal to the dosage of the active ingredient which would be administered to a subject and/or a convenient fraction of such a dosage such as, for example, one-half or one-third of such a dosage. The formulations of the disclosure can include one or more excipients and/or additives, each in an amount that together increases the stability of the viral vector, increases cell transfection or transduction by the viral vector, increases the expression of viral vector encoded protein, and/or alters the release profile of viral vector encoded proteins. In some aspects, the pharmaceutical composition comprises an excipient and/or additive. Non limiting examples of excipients and/or additives include solvents, dispersion media, diluents, or other liquid vehicles, dispersion or suspension aids, surface active agents, isotonic agents, thickening or emulsifying agents, preservatives, or combination thereof.

In some aspects, the pharmaceutical composition comprises a cryoprotectant. The term “cryoprotectant” refers to an agent capable of reducing or eliminating damage to a substance during freezing. Non-limiting examples of cryoprotectants include sucrose, trehalose, lactose, glycerol, dextrose, raffmose and/or mannitol.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

In some aspects, a pharmaceutical composition of the present disclosure can comprise phosphate-buffered saline (PBS), D-sorbitol or any combination thereof.

In some aspects, a pharmaceutical composition can comprise PBS, wherein the PBS is present at a concentration of about 100 mM to about 500 mM, or about 200 mM to about 400 mM, or about 300 mM to about 400 mM. In some aspects, the sodium chloride can be present at a concentration of about 350 mM.

In some aspects, a pharmaceutical composition can comprise D-sorbitol, wherein the D-sorbitol is present at a concentration of about 1% to about 10%, or about 2.5% to about 7.5%. In some aspects, the D-sorbitol can be present at a concentration of about 5%. Thus, the present disclosure provides a pharmaceutical composition comprising an rAAV vector and/or rAAV viral vector of the present disclosure in a 350 mM phosphate-buffered saline solution comprising D-sorbitol at a concentration of 5%.

### III. Methods

### Methods of Use

The present disclosure provides the use of a disclosed composition or pharmaceutical composition for the treatment of a disease or disorder in a cell, tissue, organ, animal, or subject, as known in the art or as described herein, using the disclosed compositions and pharmaceutical compositions, e.g., administering or contacting the cell, tissue, organ, animal, or subject with a therapeutic effective amount of the composition or pharmaceutical composition. In one aspect, the subject is a mammal. Preferably, the subject is human. The terms “subject” and “patient” are used interchangeably herein.

This disclosure provides methods of preventing or treating a disease and/or disorder, comprising, consisting essentially of, or consisting of administering to a subject a therapeutically effective amount of any one of the rAAV vectors, rAAV viral vectors, compositions and/or pharmaceutical compositions disclosed herein. The present disclosure provides the use of a composition comprising an exogenous nucleic acid in the manufacture of a medicament for a method of expressing the exogenous nucleic acid in a subject, wherein the composition is for administration to the subject.

Methods of treatment can alleviate one or more symptoms of a disease and/or disorder described herein. In an embodiment, delivery of compositions described herein can prevent or delay development of detectable symptoms, if administered to a subject carrying a mutation in the FOXG1 gene before symptoms become detectable. Therefore, treatment can be therapeutic or prophylactic. Therapy refers to inhibition or reversal of established symptoms or phenotype. Therapy can also mean delay of onset of symptoms or phenotype. Prophylaxis means inhibiting or preventing development of symptoms in subjects not already displaying overt symptoms. Subjects not displaying overt symptoms can be identified early in life as carrying a loss of function mutation in the FOXG1 gene by appropriate genetic testing performed before 18 months, 12 months, or 6 months of age.

A subject to be treated using the methods, compositions, pharmaceutical compositions, rAAV vectors or rAAV viral vectors of the present disclosure can have any of the diseases and/or symptoms described herein.

In various aspects, the disease and/or condition to be treated using the methods herein comprises FOXG1 haploinsufficiency or any disease associated with FOXG1 haploinsufficiency (e.g, West Syndrome). For example, the disease and/or condition may comprise FOXG1 syndrome, Rett-like syndrome, Autism (or autism spectrum disorder like phenotypes), West syndrome, epilepsy, cognitive or developmental delay, or any combination thereof. In some aspects the disease and/or condition comprises FOXG1 syndrome. In some aspects, the disease and/or condition comprises West syndrome. In various aspects, the methods herein allow for controlled expression of FOXG1 in the subject and avoids toxic overexpression.

The disclosure provides methods of increasing the level of a protein in a host cell, comprising contacting the host cell with any one of the rAAV viral vectors disclosed herein, wherein the rAAV viral vectors comprises any one of the rAAV vectors disclosed herein, comprising a transgene nucleic acid molecule encoding the protein. In some aspects, the protein is a therapeutic protein. In some aspects, the host cell is in vitro, in vivo, or ex vivo. In some aspects, the host cell is derived from a subject. In some aspects, the subject suffers from a disorder, which results in a reduced level and/or functionality of the protein, as compared to the level and/or functionality of the protein in a normal subject.

In some aspects, the level of the protein is increased to level of about 1×10⁷ ng, about 3×10⁷ ng, about 5×10⁷ ng, about 7×10⁷ ng, about 9×10⁷ ng, about 1×10⁶ ng, about 2×10⁶ ng, about 3×10⁶ ng, about 4×10⁶ ng, about 6×10⁶ ng, about 7×10⁶ ng, about 8×10⁶ ng, about 9×10⁶ ng, about 10×10⁶ ng, about 12×10⁶ ng, about 14×10⁶ ng, about 16×10⁶ ng, about 18×10⁻⁶ ng, about 20×10⁶ ng, about 25×10⁶ ng, about 30×10⁶ ng, about 35×10⁶ ng, about 40×10⁶ ng, about 45×10⁶ ng, about 50×10⁶ ng, about 55×10⁶ ng, about 60×10⁶ ng, about 65×10⁻⁶ ng, about 70×10⁶ ng, about 75×10⁶ ng, about 80×10⁶ ng, about 85×10⁶ ng, about 90×10⁶ ng, about 95×10⁶ ng, about 10×10⁵ ng, about 20×10⁵ ng, about 30×10⁵ ng, about 40×10⁵ ng, about 50×10⁵ ng, about 60×10⁵ ng, about 70×10⁵ ng, about 80×10⁵ ng, or about 90×10⁵ ng in the host cell.

In some cases, the level of protein is increased, but not increased to the level observed in a cell treated with an equivalent vector lacking an miRNA regulatory element. For example, in some cases, the protein is expressed at a level that is about 20%, 30%, 40%, 50%, 60%, 70%, or 80% the level achieved in a cell comprising an equivalent vector lacking an miRNA regulatory element. For example, in some cases, the protein is expressed at a level that is about 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% the level achieved in a cell comprising an equivalent vector lacking an miRNA regulatory element.

The disclosure provides methods of introducing a gene of interest to a cell in a subject comprising contacting the cell with an effective amount of any one of the rAAV viral vectors disclosed herein, wherein the rAAV viral vectors contain any one of the rAAV vectors disclosed herein, comprising the gene of interest.

In some aspects of the methods of the present disclosure, a subject can also be administered a prophylactic immunosuppressant treatment regimen in addition to being administered an rAAV vector or rAAV viral vector of the present disclosure. In some aspects, an immunosuppressant treatment regimen can comprise administering at least one immunosuppressive therapeutic. Non limiting examples of immunosuppressive therapeutics include, but are not limited to, Sirolimus (rapamycin), acetaminophen, diphenhydramine, IV methylprednisolone, prednisone, or any combination thereof. An immunosuppressive therapeutic can be administered prior to the day of administration of the rAAV vector and/or rAAV viral vector, on the same day as the administration of the rAAV vector and/or rAAV viral vector, or any day following the administration of the rAAV vector and/or rAAV viral vector.

As used herein the term “effective amount” and/or “therapeutically effective amount” intends to mean a quantity sufficient to achieve a desired effect. In the context of therapeutic or prophylactic applications, the effective amount will depend on the type and severity of the condition at issue and the characteristics of the individual subject, such as general health, age, sex, body weight, and tolerance to pharmaceutical compositions. In the context of gene therapy, the effective amount can be the amount sufficient to result in regaining part or full function of a gene that is deficient in a subject. In some aspects, the effective amount of an rAAV viral vector is the amount sufficient to result in expression of a gene in a subject such that a FOXG1 polypeptide is produced. In some aspects, the effective amount is the amount required to increase galactose metabolism in a subject in need thereof. The skilled artisan will be able to determine appropriate amounts depending on these and other factors.

In some aspects, the effective amount will depend on the size and nature of the application in question. It will also depend on the nature and sensitivity of the target subject and the methods in use. The skilled artisan will be able to determine the effective amount based on these and other considerations. The effective amount may comprise, consist essentially of, or consist of one or more administrations of a composition depending on the embodiment.

As used herein, the term “administer” or “administration” intends to mean delivery of a substance to a subject such as an animal or human. Administration can be effected in one dose, continuously or intermittently throughout the course of treatment. Methods of determining the most effective means and dosage of administration are known to those of skill in the art and will vary with the composition used for therapy, the purpose of the therapy, as well as the age, health or gender of the subject being treated. Single or multiple administrations can be carried out with the dose level and pattern being selected by the treating physician or in the case of pets and other animals, treating veterinarian.

Methods of determining the most effective means and dosage of administration are known to those of skill in the art and will vary with the composition used for therapy, the purpose of the therapy and the subject being treated. Single or multiple administrations can be carried out with the dose level and pattern being selected by the treating physician. It is noted that dosage may be impacted by the route of administration. Suitable dosage formulations and methods of administering the agents are known in the art. Non-limiting examples of such suitable dosages may be as low as 10⁹ vector genomes to as much as 10¹⁷ vector genomes per administration.

In some aspects of the methods described herein, the number of viral particles (e.g., rAAV viral vectors) administered to the subject ranges from about 10⁹ to about 10¹⁷. In some aspects, about 10¹⁰ to about 10¹², about 10¹¹ to about 10¹³, about 10¹¹ to about 10¹², about 10¹¹ to about 10¹⁴, about 10¹² to about 10¹⁶, about 10¹³ to about 10¹⁶, about 10¹⁴ to about 10¹⁵, about 5×10¹¹ to about 5×10¹², about 10¹¹ to about 10¹⁸, about 10¹³ to about 10¹⁶, or about 10¹² to about 10¹³ viral particles are administered to the subject.

In some aspects of the methods described herein, the number of viral particles (e.g., rAAV viral vectors) administered to the subject is at least about 10¹⁰, or at least about 10¹¹, or at least about 10¹², or at least about 10¹³, or at least about 10¹⁴, or at least about 10¹⁵, or at least about 10¹⁶, or at least about 10¹⁷ viral particles.

In some aspects of the methods described herein the number of viral particles (e.g. rAAV viral vectors) administered to the subject is about 3.5×10¹³ viral particles. In some aspects of the methods described herein the number of viral particles (e.g. rAAV viral vectors) administered to the subject is about 3.5×10¹⁴ viral particles. In some aspects of the methods described herein the number of viral particles (e.g. rAAV viral vectors) administered to the subject is about 3.5×10¹³ to about 3.5×10¹⁴ viral particles.

In some aspects of the methods described herein, the number of viral particles (e.g., rAAV viral vectors) administered to the subject can depend on the age of the subject. In non-limiting examples, a subject that is 7 years of age or older can be administered about 10×10¹⁴ viral particles, a subject that is about 4 years of age to about 7 years of age can be administered about 10×10¹⁴ viral particles, a subject that is about 3 years of age to about 4 years of age can be administered about 9×10¹⁴ viral particles, a subject that is about 2 years of age to about 3 years of age can be about 8.2×10¹⁴ viral particles, a subject that is about 1 year of age to about 2 years of age can be administered about 7.3×10¹⁴ viral particles, a subject that is about 0.5 years of age to about 1 year of age can be administered about 4×10¹⁴ viral particles, or a subject that is less than 0.5 years of age can be administered 3×10¹⁴ viral particles.

In some aspects, the amounts of viral particles in a composition, pharmaceutical composition, or the amount of viral particles administered to a patient can calculated based on the percentage of viral particles that are predicted to contain viral genomes.

In some aspects, rAAV viral vectors of the present disclosure can be introduced to the subject intravenously, intrathecally, intracerebrally, intraventricularly, intranasally, intratracheally, intra-aurally, intra-ocularly, or peri-ocularly, orally, rectally, transmucosally, inhalationally, transdermally, parenterally, subcutaneously, intradermally, intramuscularly, intracistemally, intranervally, intrapleurally, topically, intralymphatically, intracistemally; such introduction may also be intra-arterial, intracardiac, subventricular, epidural, intracerebral, intracerebroventricular, sub-retinal, intravitreal, intraarticular, intraperitoneal, intrauterine, intranerve or any combination thereof. In some aspects, the viral particles are delivered to a desired target tissue, e.g., to the lung, eye, or CNS, as non-limiting examples. In some aspects, delivery of viral particles is systemic. The intracisternal route of administration involves administration of a drug directly into the cerebrospinal fluid of the brain ventricles. It could be performed by direct injection into the cisterna magna or via a permanently positioned tube. In some aspects, the rAAV viral vectors of the present disclosure are administered intrathecally.

In some aspects, the rAAV viral vectors of the present disclosure repair a gene deficiency in a subject. In some aspects, the ratio of repaired target polynucleotide or polypeptide to unrepaired target polynucleotide or polypeptide in a successfully treated cell, tissue, organ or subject is at least about 1.5:1, about 2:1, about 3:1, about 4:1, about 5:1, about 6:1, about 7:1, about 8:1, about 9:1, about 10:1, about 20:1, about 50:1, about 100:1, about 1000:1, about 10,000:1, about 100,000:1, or about 1,000,000:1. The amount or ratio of repaired target polynucleotide or polypeptide can be determined by any method known in the art, including but not limited to western blot, northern blot, Southern blot, PCR, sequencing, mass spectrometry, flow cytometry, immunohistochemistry, immunofluorescence, fluorescence in situ hybridization, next generation sequencing, immunoblot, and ELISA.

Administration of the rAAV vectors, rAAV viral vectors, compositions or pharmaceutical compositions of this disclosure can be effected in one dose, continuously or intermittently throughout the course of treatment. In some aspects, the rAAV vectors, rAAV viral vectors, compositions, or pharmaceutical compositions of this disclosure are parenterally administered by injection, infusion, or implantation.

In some aspects, the rAAV viral vectors of this disclosure show enhanced tropism for brain and cervical spine. In some aspects, the rAAV viral vectors of the disclosure can cross the blood-brain-barrier (BBB).

Suitable subjects may include, without limit, humans, as well as companion animals such as cats, dogs, rodents, and horses; research animals such as rabbits, sheep, pigs, dogs, primates, mice, rats, and other rodents; agricultural animals such as cows, cattle, pigs, goats, sheep, horses, deer, chickens, and other fowl; zoo animals; and primates such as chimpanzees, monkeys, and gorillas. The subject can be of any age without limitation. In an aspect, the subject may be a human.

### Methods of Manufacture

A variety of approaches may be used to produce rAAV viral vectors of the present disclosure. In some aspects, packaging is achieved by using a helper virus or helper plasmid and a cell line. The helper virus or helper plasmid contains elements and sequences that facilitate viral vector production. In another aspect, the helper plasmid is stably incorporated into the genome of a packaging cell line, such that the packaging cell line does not require additional transfection with a helper plasmid.

In some aspects, the cell is a packaging or helper cell line. In some aspects, the helper cell line is eukaryotic cell; for example, an HEK 293 cell or 293T cell. In some aspects, the helper cell is a yeast cell or an insect cell.

In some aspects, the cell comprises a nucleic acid encoding a tetracycline activator protein; and a promoter that regulates expression of the tetracycline activator protein. In some aspects, the promoter that regulates expression of the tetracycline activator protein is a constitutive promoter. In some aspects, the promoter is a phosphoglycerate kinase promoter (PGK) or a CMV promoter.

A helper plasmid may comprise, for example, at least one viral helper DNA sequence derived from a replication-incompetent viral genome encoding in trans all virion proteins required to package a replication incompetent AAV, and for producing virion proteins capable of packaging the replication-incompetent AAV at high titer, without the production of replication-competent AAV.

Helper plasmids for packaging AAV are known in the art, see, e.g., U.S. Patent Pub. No. 2004/0235174 AI, incorporated herein by reference. As stated therein, an AAV helper plasmid may contain as helper virus DNA sequences, by way of non-limiting example, the Ad5 genes E2A, E4 and VA, controlled by their respective original promoters or by heterologous promoters. AAV helper plasmids may additionally contain an expression cassette for the expression of a marker protein such as a fluorescent protein to permit the simple detection of transfection of a desired target cell.

The disclosure provides methods of producing rAAV viral vectors comprising transfecting a packaging cell line with any one of the AAV helper plasmids disclosed herein; and any one of the rAAV vectors disclosed herein. In some aspects, the AAV helper plasmid and rAAV vector are co-transfected into the packaging cell line. In some aspects, the cell line is a mammalian cell line, for example, human embryonic kidney (HEK) 293 cell line. The disclosure provides cells comprising any one of the rAAV vectors and/or rAAV viral vectors disclosed herein.

As used herein, the term “helper” in reference to a virus or plasmid refers to a virus or plasmid used to provide the additional components necessary for replication and packaging of any one of the rAAV vectors disclosed herein. The components encoded by a helper virus may include any genes required for virion assembly, encapsidation, genome replication, and/or packaging. For example, the helper virus or plasmid may encode necessary enzymes for the replication of the viral genome. Non-limiting examples of helper viruses and plasmids suitable for use with AAV constructs include pHELP (plasmid), adenovirus (virus), or herpesvirus (virus). In some aspects, the pHELP plasmid may be the pHELPK plasmid, wherein the ampicillin expression cassette is exchanged with a kanamycin expression cassette.

As used herein, a packaging cell (or a helper cell) is a cell used to produce viral vectors. Producing recombinant AAV viral vectors requires Rep and Cap proteins provided in trans as well as gene sequences from Adenovirus that help AAV replicate. In some aspects, Packaging/helper cells contain a plasmid is stably incorporated into the genome of the cell. In other aspects, the packaging cell may be transiently transfected. Typically, a packaging cell is a eukaryotic cell, such as a mammalian cell or an insect cell.

### IV. Kits

The isolated polynucleotides, rAAV vectors, rAAV viral vectors, compositions, and/or pharmaceutical compositions described herein may be assembled into pharmaceutical or diagnostic or research kits to facilitate their use in therapeutic, diagnostic, or research applications. In some aspects, the kits of the present disclosure include any one of the isolated polynucleotides, rAAV vectors, rAAV viral vectors, compositions, pharmaceutical compositions, host cells, isolated tissues, as described herein.

In some aspects, a kit further comprises instructions for use. Specifically, such kits may include one or more agents described herein, along with instructions describing the intended application and the proper use of these agents. In some aspects, the kit may include instructions for mixing one or more components of the kit and/or isolating and mixing a sample and applying to a subject. In some aspects, agents in a kit are in a pharmaceutical formulation and dosage suitable for a particular application and for a method of administration of the agents. Kits for research purposes may contain the components in appropriate concentrations or quantities for running various experiments.

The kit may be designed to facilitate use of the methods described herein and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. In some aspects, the compositions may be provided in a preservation solution (e.g., cryopreservation solution). Non-limiting examples of preservation solutions include DMSO, paraformaldehyde, and CryoStor® (Stem Cell Technologies, Vancouver, Canada). In some aspects, the preservation solution contains an amount of metalloprotease inhibitors.

In some aspects, the kit contains any one or more of the components described herein in one or more containers. Thus, in some aspects, the kit may include a container housing agents described herein. The agents may be in the form of a liquid, gel or solid (powder). The agents may be prepared sterilely, packaged in a syringe and shipped refrigerated. Alternatively, they may be housed in a vial or other container for storage. A second container may have other agents prepared sterilely. Alternatively, the kit may include the active agents premixed and shipped in a syringe, vial, tube, or other container. The kit may have one or more or all of the components required to administer the agents to a subject, such as a syringe, topical application devices, or IV needle tubing and bag.

### V. Examples

### Example 1: Background and Summary of Examples

Loss-of-function and duplication of the transcription regulator FOXG1 mediate FOXG1 haploinsufficiency syndrome and West syndrome respectively. The role of FOXG1 in patterning the embryonic telencephalon is well characterized, but relatively little is known about postnatal FOXG1 activity. FOXG1 haploinsufficiency syndrome is characterized by intellectual disability and seizures. West syndrome is also characterized by seizures. FOXG1 syndrome may therefore be a good candidate for a regulated gene therapy that permits efficacy without dose-dependent toxicity.

The urgency for initiating a gene therapy program is strong, as patients have no FDA-approved genetic intervention. To address this urgency, the inventors evaluated both unregulated and regulated FOXG1 vectors to determine if the regulated vector circumvents side effects of the unregulated vector. It would be reasonable to hypothesize, for example, that the unregulated vector increases miRNAs associated with seizures, which is one of the symptoms of West Syndrome. As described in the Examples below, the regulatory element miRARE (MicroRNA-Responsive Autoregulatory Element) was inserted into the 3′UTR of the FOXG1 viral genome to create the regulated construct. The present disclosure seeks to resolve the supraphysiological cellular overexpression of FOXG1 previously documented for AAV9-mediated RNA activation, an alternative approach recently proposed for treating FOXG1 syndrome. In lieu of miRARE, an alternative panel of miRNA binding sites can also be designed from the miRNA expression data described herein.

### Example 2: Preparation of FOXG1 Gene Therapy Vectors

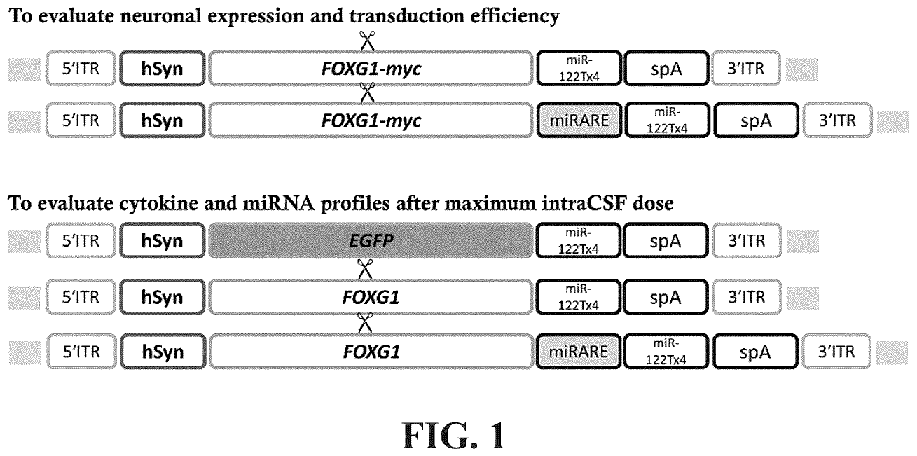

The inventors prepared gene therapy vectors as depicted in FIG. 1. The viral genome designs shown above were each packaged in single-stranded (ss) AAV9. hSyn, human synapsin promoter; ITR, inverted terminal repeats; miR-122Tx4, four tandem binding sites (or targets) for the hepatic miR-122; miRARE, miR-responsive auto-regulatory element; spA, synthetic poly A. MiR-122Tx4 is a courtesy safety feature. Together, the promoter and the hepatic miRNA binding sites were intended to prevent hepatic expression of FOXG1±myc. The scissors indicate where a proteolytic cleavage site is encoded.

Tables 1-4 below provide a description of the elements included in the four vectors generated (SEQ ID NOs: 16-19).

TABLE 1
FOXG1 (untagged) (ITR-hSyn-hFOXG1-
STOP-liverT-spA-ITR, SEQ ID NO: 16)

| | Location | SEQ |
|---|---|---|
| Element | (nt . . . nt) | ID NO: |
| 5′ ITR | 1 . . . 168 | 1 |
| Alpha-synuclein promoter | 175 . . . 622 | 3 |
| FOXG1 transgene with Kozak sequence | 623 . . . 2102 | 4 |
| miR-122 Regulatory Binding Site Panel | 2118 . . . 2217 | 12 |
| (LiverT) | | |
| spA | 2224 . . . 2271 | 13 |
| 3′ ITR | 2291 . . . 2458 | 14 |

TABLE 2
FOXG1-myc with Linker (ITR-hSyn-hFOXG1-LINKER-
myc-STOP-liverT-spA-ITR, SEQ ID NO: 17)

| | Location | SEQ |
|---|---|---|
| Element | (nt . . . nt) | ID NO: |
| 5′ ITR | 1 . . . 168 | 1 |
| Alpha-synuclein promoter | 175 . . . 622 | 3 |
| FOXG1 transgene preceded by Kozak sequence | 623 . . . 2102 | 4 |
| Flexible Linker + Myc | 2103 . . . 2153 | 8 |
| miR-122 Regulatory Binding Site Panel | 2169 . . . 2268 | 12 |
| (LiverT) | | |
| spA | 2275 . . . 2322 | 13 |
| 3′ ITR | 2342 . . . 2509 | 14 |

TABLE 3
FOXG1-myc-miRARE with Linker (ITR-hSyn-hFOXG1-LINKER-
myc-STOP-miRARE-liverT-spA-ITR, SEQ ID NO: 18)

| | Location | SEQ |
|---|---|---|
| Element | (nt . . . nt) | ID NO: |
| 5′ ITR | 1 . . . 168 | 1 |
| Alpha-synuclein promoter | 175 . . . 622 | 3 |
| FOXG1 transgene with Kozak sequence | 623 . . . 2102 | 4 |
| Flexible Linker + Myc | 2103 . . . 2153 | 8 |
| miRARE | 2163 . . . 2337 | 11 |
| miR-122 Regulatory Binding Site Panel | 2169 . . . 2268 | 12 |
| (LiverT) | | |
| spA | 2450 . . . 2497 | 13 |
| 3′ ITR | 2517 . . . 2684 | 14 |

TABLE 4
FOXG1-myc-miRARE (ITR-hSyn-hFOXG1-STOP-
miRARE-liverT-spA-ITR, SEQ ID NO: 19)

| | Location | SEQ |
|---|---|---|
| Element | (nt . . . nt) | ID NO: |
| 5′ ITR | 1 . . . 168 | 1 |
| Alpha-synuclein promoter | 175 . . . 622 | 3 |
| FOXG1 transgene with Kozak sequence | 623 . . . 2102 | 4 |
| miRARE | 2112 . . . 2286 | 11 |
| miR-122 Regulatory Binding Site Panel | 2293 . . . 2392 | 12 |
| (LiverT) | | |
| spA | 2399 . . . 2446 | 13 |
| 3′ ITR | 2466 . . . 2633 | 14 |

In the following examples, AAV9 vectors were administered to mice to either: (1) evaluate neuronal expression and transduction efficiency; or (2) evaluate cytokine and miRNA profiles after a high dose into the cerebrospinal fluid. Male wild-type (WT) mice were injected into the cisterna magna (ICM) at P28-35 with a FOXG1 (±myc) vector. One-month post-injection, animals were euthanized, and samples were collected for analysis.

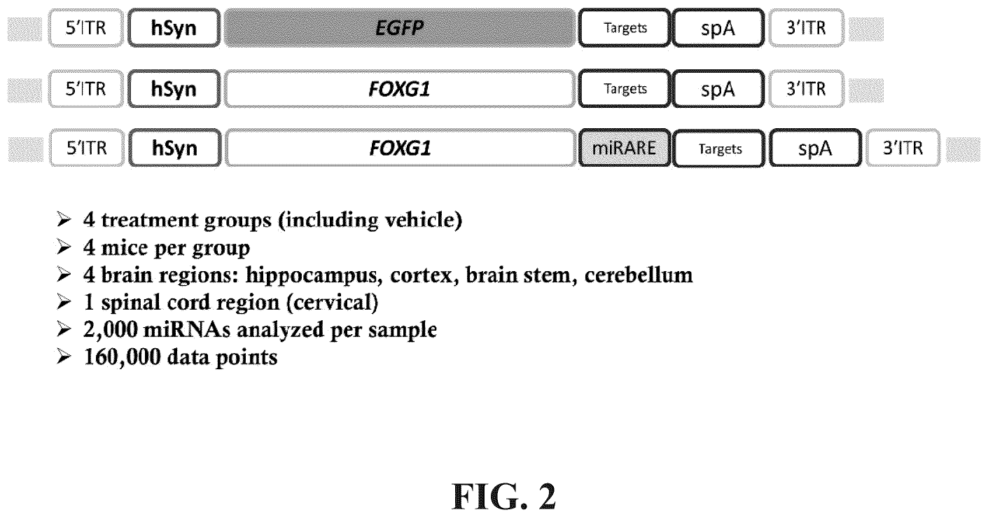

In a first experiment, the expression levels of approximately 2,000 mouse miRNAs were quantified from tissues from mice treated with FOXG1 or EGFP control vectors. Viral genome designs for vectors used in the in vivo study are illustrated in FIG. 2. Mice received a high intraCSF dose to model an overdose of an unregulated gene therapy as described in Sinnett et al., Brain 2021; 144 (1)):3005-3019 which is incorporated herein by reference in its entirety. As described in FIG. 2, 4 treatment groups were included (including vehicle) with 4 mice per group. 4 brain regions: hippocampus, cortex, brain stem, and cerebellum, and one spinal cord region (cervical) were analyzed. A total of 2,000 miRNAs were analyzed per sample for a total of 160,000 datapoints.

### Example 2: RAAV Delivery Leads to FOXG1 Expression in Neurons

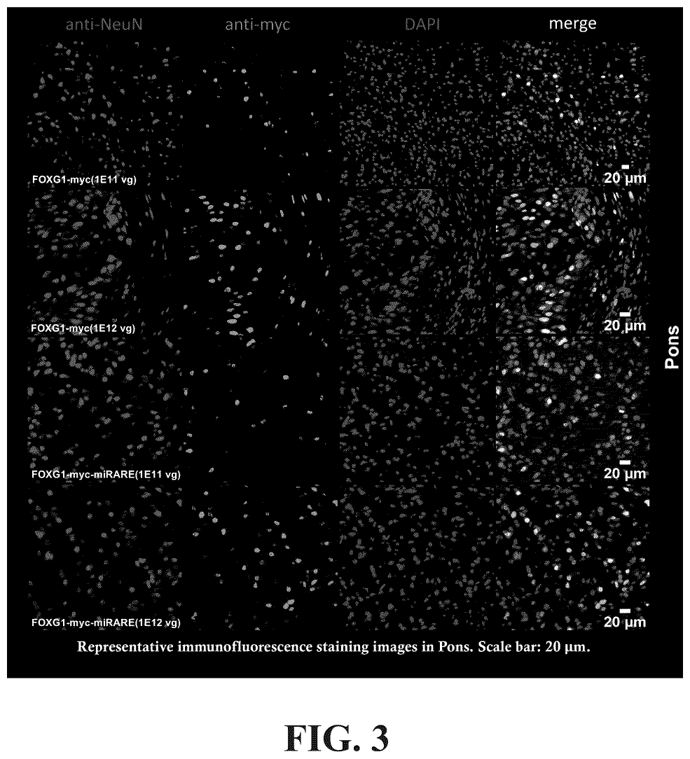

Following intraCSF injection of low (1E11 vg/mouse) or high (1E12 vg/mouse) levels of FOXG1-myc (SEQ ID NO: 17) or FOXG1-myc-miRARE (SEQ ID NO: 18) vectors the expression of FOXG1 (as measured by myc (+) cells)) was determined using immunofluorescence in the pons of treated mice. It was found that most myc (+) cells in the brain of treated mice were neuronal and miRARE had no effect on this observation (FIG. 3).

### Example 3: AAV9/hFOXG1-Myc-miRARE Treated Mice had Suppressed FOXG1 Expression Compared to AAV9/hFOXG1 Treated Mice

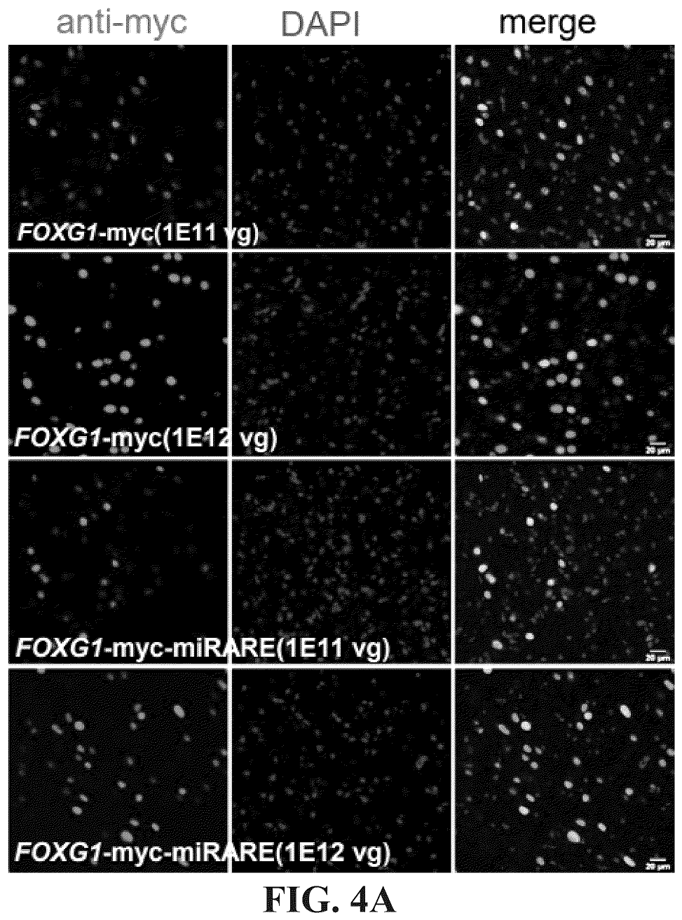

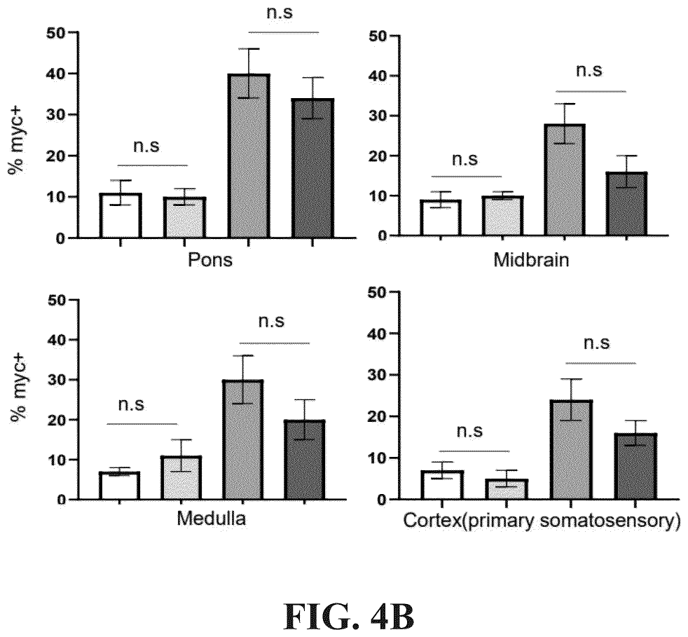

In another experiment, mice were treated with low (1E11 vg/mouse) or high (1E12 vg/mouse) AAV9/hFOXG1-myc-miRARE (SEQ ID NO: 18) and AAV9/hFOXG1 (SEQ ID NO: 17) vectors were analyzed for neuronal expression of FOXG1 (measured by detecting the myc tag) in various brain regions (pons, midbrain, medulla, and cortex. FIG. 4A shows representative immunofluorescence staining images of myc expression in midbrain after different treatments. FIG. 4B summarizes levels of myc in different brain regions and shows that AAV9/hFOXG1-myc-miRARE-treated mice exhibited a trend toward suppressing FOXG1 expression compared with AAV9/hFOXG1-treated mice in a dose-dependent manner.

### Example 4: RAAV Treatment does not Induce Expression of FOXG1 in Cerebellum

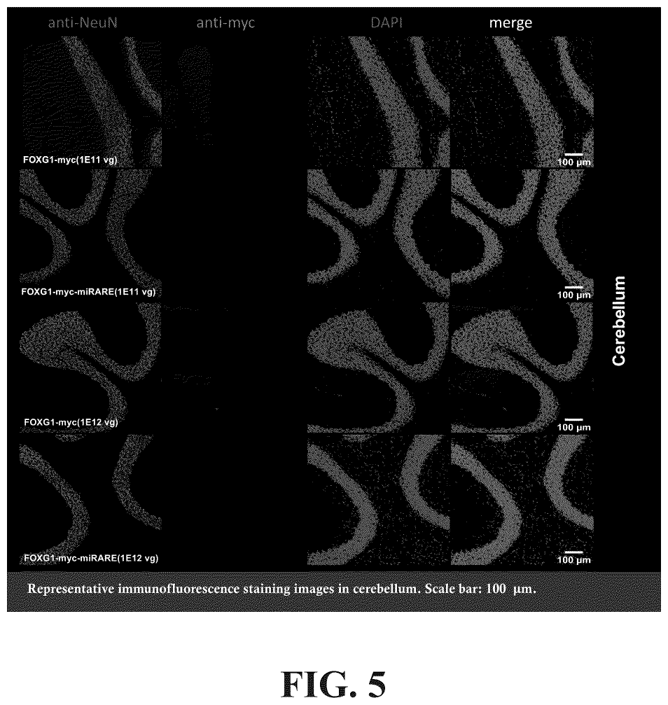

In another experiment, mice were treated via intraCSF injection with low (1E11 vg/mouse) or high (1E12 vg/mouse) AAV9/hFOXG1-myc-miRARE (SEQ ID NO: 18) and AAV9/hFOXG1 (SEQ ID NO: 17) vectors and FOXG1 expression (as measured by myc-tag) was measured in the cerebellum. As shown in FIG. 5, no myc-tagged protein was observed in the cerebellum and miRARE had no effect on this observation. The absence of FOXG1-myc protein is noteworthy because the exogenous human FOXG1 gene is detectable in the cerebellum (data not shown).

### Example 5: AAV Treatment does not Alter Normal Peripheral Cytokine Levels

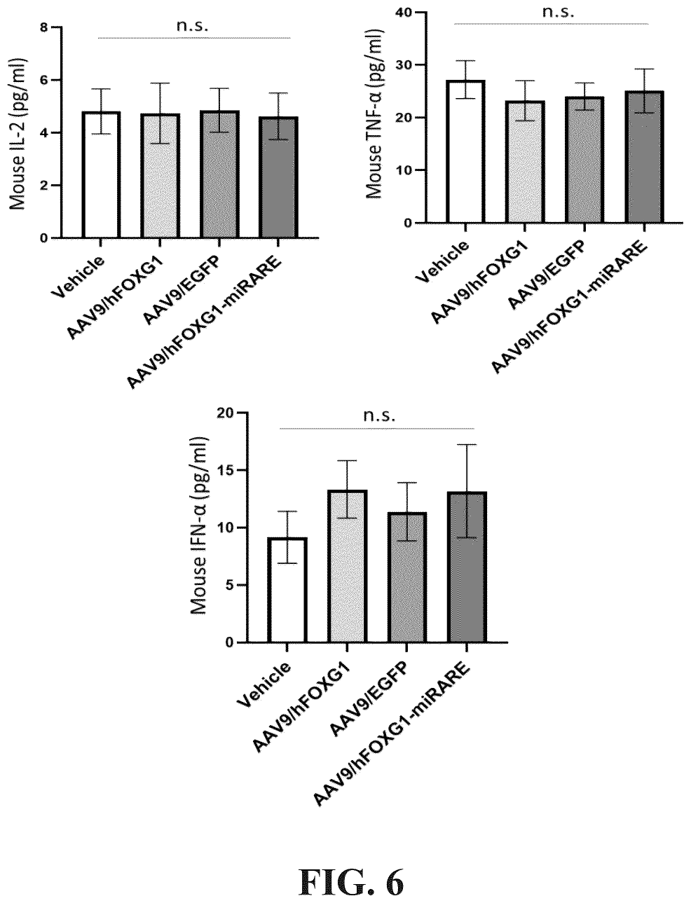

In another experiment, mice were treated with a AAV9/hFOXG1 vector (SEQ ID NO: 17), a AAV9/hFOXG1-myc-miRARE vector (SEQ ID NO: 18), a control AAV9/EGFP vector, or vehicle and levels of IL-2, TNF-α and IFN-α were measured in the serum using ELISA. It was found that treatment with the AAV vectors did not significantly change peripheral cytokine profiles (FIG. 6).

### Example 6: The Unregulated FOXG1 Vector Significantly Changed the Expression of Many miRNAs

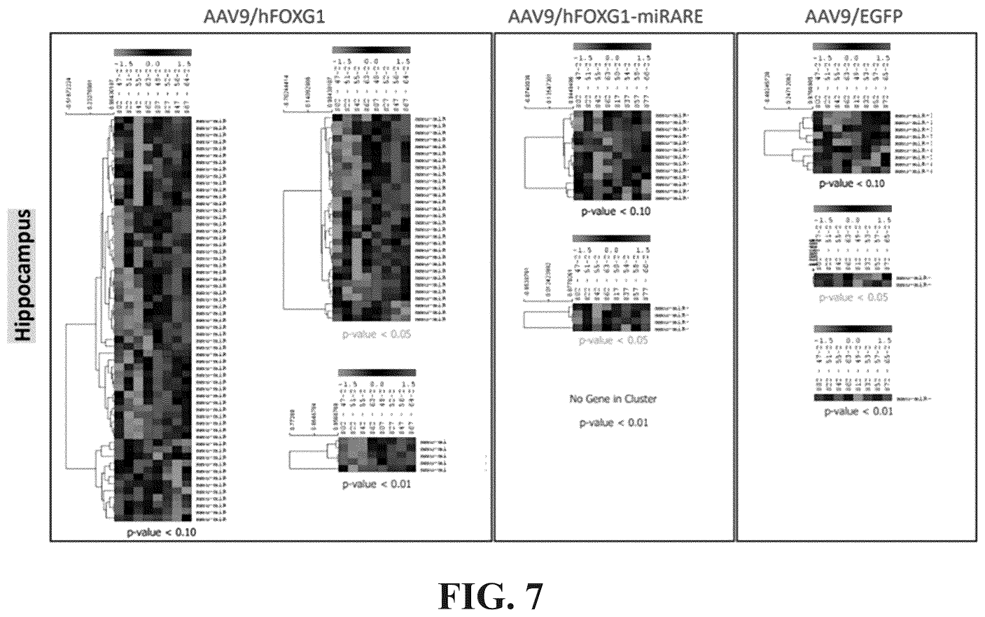

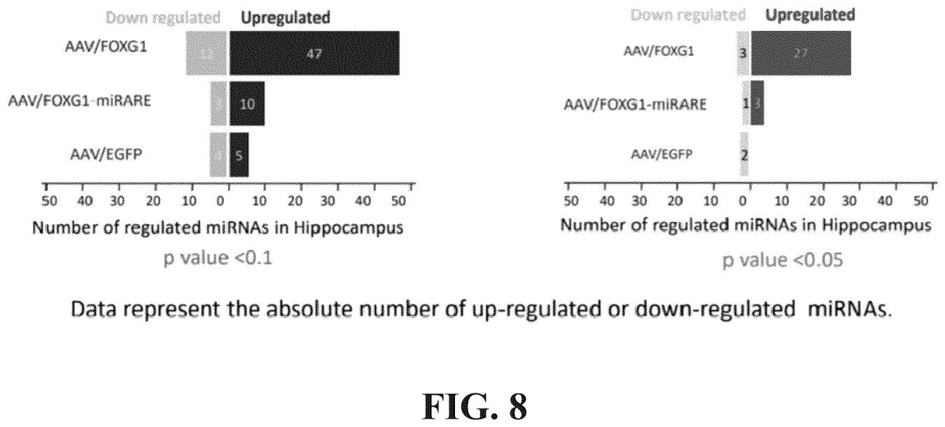

In another experiment, miRNA expression following treatment with a AAV9/hFOXG1 vector (SEQ ID NO: 17), a AAV9/hFOXG1-myc-miRARE vector (SEQ ID NO: 18), a control AAV9/EGFP vector was measured using microarrays. As shown in FIG. 7 and FIG. 8, the unregulated FOXG1 vector (AAV9/hFOXG1 vector (SEQ ID NO: 17)) significantly changed the expression of many miRNAs in the hippocampus of treated animals. This observation is important because the regulated FOXG1 vector (AAV9/hFOXG1-myc-miRARE vector (SEQ ID NO: 18)) and the EGFP vector appeared to be well-tolerated and significantly changed the expression of few hippocampal miRNAs. It is unclear if the miRNA expression profile in AAV9/hFOXG1-treated mice indicates damage or perhaps a protective effect that attenuates damage. However, half of the upregulated miRNAs are known to be upregulated in animal models of seizures and/or human epilepsy. It was also found that vectors had an unremarkable effect on the miRNA expression profile of the cerebellum (data not shown).

### Example 7: Untreated FOXG1Cre/+ Mice Exhibit Anxiety Related Behavior in an Open Field Test

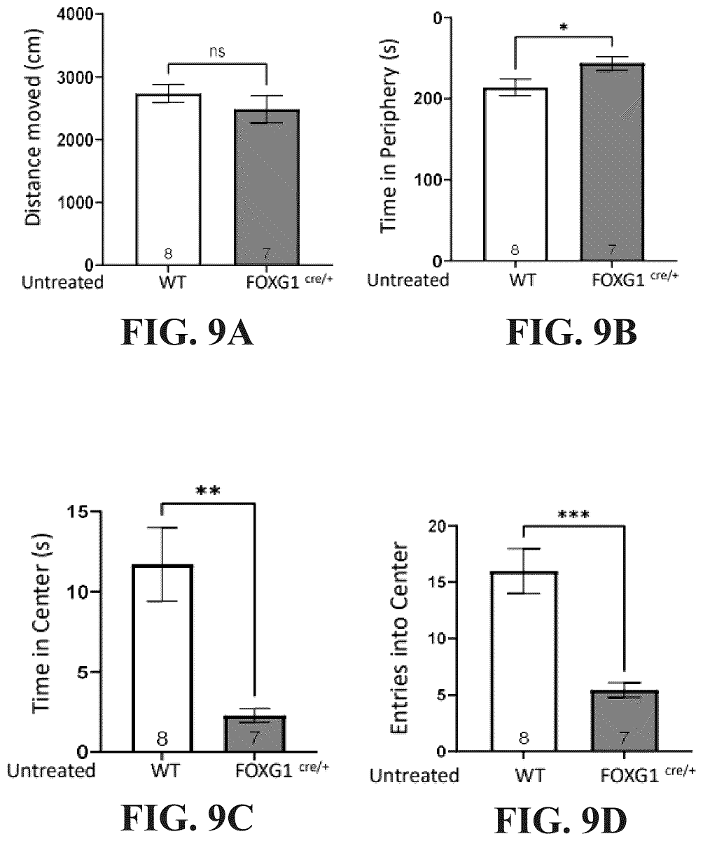

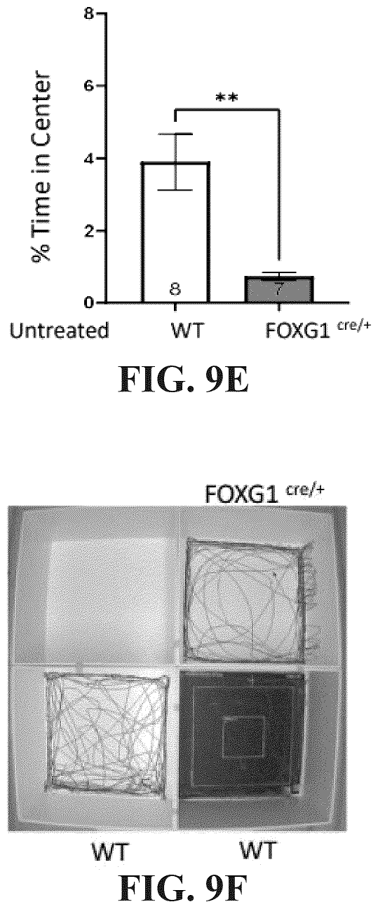

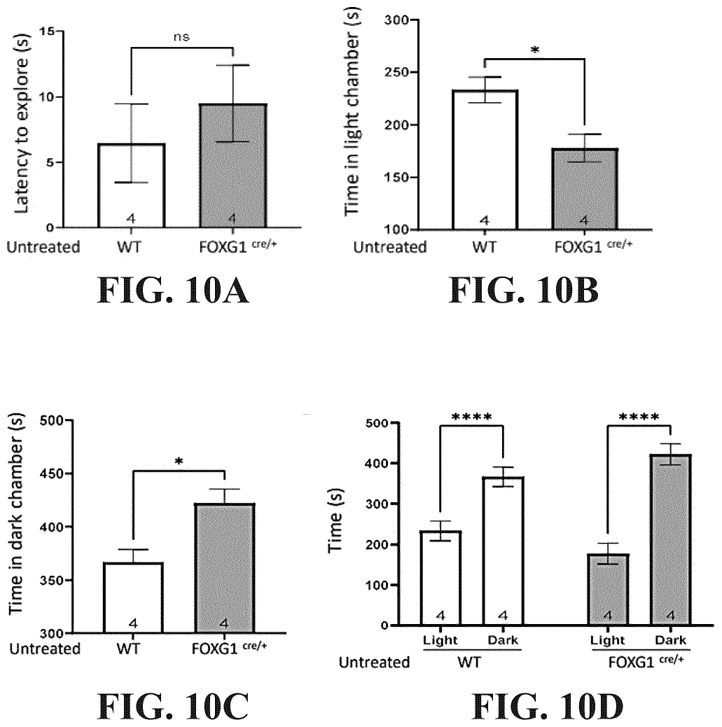

To establish a mouse model for FOXG1 haploinsufficiency, a B6.129P2 (Cg)-FOXG1tm1 (cre) Skm/J mice (Strain #: 006084, FOXG1 cre/+) was generated at Jackson Laboratory and tested for a behavioral phenotype. As shown in FIGS. 9A-9F untreated FOXG1cre/+ mice having a reduced expression of FOXG1 exhibited anxiety related behavior in an open field test. As shown in FIGS. 10A-10D, untreated FOXG1cre/+ mice having a reduced expression of FOXG1 exhibited increased anxiety in the dark/light preference test. Therefore, these mice can be used, then, to test the behavioral effect of restoring FOXG1 expression in vivo using the vectors of the present disclosure.

### Example 8: Behavioral Outcomes of Treating FOXG1Cre/+ Mice with Regulated and Unregulated AAV Vectors

In an experiment, FOXG1cre/+ mice will be treated with vehicle, low (1E11 vg/mouse) or high (1E12 vg/mouse) levels of AAV9/hFOXG1-myc-miRARE (SEQ ID NO: 18) and AAV9/hFOXG1 (SEQ ID NO: 17) vectors and measured for behavioral changes using the behavioral assays described in Example 7. Specifically, mice will be analyzed for anxiety using the open field test shown in FIGS. 9A-9F and the light/dark preference test as shown in FIGS. 10A-10D. It is predicted that FOXG1cre/+ mice treated with regulated vectors (e.g., SEQ ID NO: 18) will show improved behavioral outcomes relative to mice treated with unregulated vectors (e.g., SEQ ID NO: 17).

What is claimed is:

1. An expression construct comprising a transgene nucleic acid encoding FOXG1 and one or more miRNA regulatory elements.

2. The expression construct claim 1, wherein the one or more miRNA regulatory elements comprise a micro-RNA responsive autoregulatory element (miRARE).

3. The expression construct of claim 2, wherein the miRARE comprises a nucleic acid sequence having at least 70%, at least 75%, at least 80%, at least 85%, at least 90%, at least 95%, at least 96%, at least 97%, at least 98%, at least 99%, or 100% sequence identity to SEQ ID NO: 11.

4. The expression construct of claim 1, wherein one or more miRNA regulatory elements comprise one or more miRNA binding sites.

5. The expression construct of claim 4, wherein the one or more miRNA regulatory elements comprise one or more binding sites for a tissue specific miRNA.

6. The expression construct of claim 5, wherein the one or more miRNA regulatory elements comprise a nucleic acid sequence having at least 70%, at least 75%, at least 80%, at least 85%, at least 90%, at least 95%, at least 96%, at least 97%, at least 98%, at least 99%, or 100% sequence identity to SEQ ID NO: 12.

7. The expression construct of claim 1, wherein the one or more miRNA regulatory elements comprise a nucleic acid sequence having at least 70%, at least 75%, at least 80%, at least 85%, at least 90%, at least 95%, at least 96%, at least 97%, at least 98%, at least 99%, or 100% sequence identity to SEQ ID NO: 11 and a nucleic acid sequence having at least 70%, at least 75%, at least 80%, at least 85%, at least 90%, at least 95%, at least 96%, at least 97%, at least 98%, at least 99%, or 100% sequence identity to SEQ ID NO: 12.

8. The expression construct of claim 1, wherein the one or more miRNA regulatory elements trigger miRNA directed degradation of a FOXG1 RNA transcript encoded by the expression construct.

9. The expression construct of claim 8, wherein the miRNA directed degradation is tissue specific.

10. The expression construct of claim 1, wherein the expression construct comprises a viral vector.

11. The expression construct of claim 10, wherein the viral vector comprises a recombinant adeno-associated viral vector (rAAV vector).

12. The expression construct of claim 11, wherein the rAAV vector comprises in a 5′ to 3′ direction a) a first AAV ITR sequence; b) a promoter sequence; c) a transgene nucleic acid molecule, wherein the transgene nucleic acid molecule comprises a nucleic acid sequence encoding for a FOXG1 polypeptide; d) a miRNA regulatory element, e) a polyA sequence; and f) a second AAV ITR sequence.

13. The expression construct of claim 12, wherein the first AAV ITR sequence comprises SEQ ID NO: 1 or 2 and/or the second AAV ITR sequence comprises SEQ ID NO: 14 or 15.

14. The expression construct of claim 12, wherein the promoter sequence comprises SEQ ID NO: 3.

15. The expression construct of claim 12, wherein the miRNA regulatory element comprises SEQ ID NO: 11.

16. The expression construct of claim 12, wherein the FOXG1 polypeptide comprises an amino acid sequence of SEQ ID NO: 5.

17. The expression construct of claim 15, wherein the AAV vector comprises a nucleic acid sequence having at least 70%, at least 75%, at least 80%, at least 85%, at least 90%, at least 95%, at least 96%, at least 97%, at least 98%, at least 99%, or 100% sequence identity to one ore more of SEQ ID NOs: 16 to 18.

18. A pharmaceutical composition comprising an expression construct of claim 1 and at least one pharmaceutically appropriate carrier or excipient.

19. A method of treating FOXG1 haploinsufficiency in a subject in need thereof, the method comprising administering a therapeutically effective amount of the pharmaceutical composition of claim 18 to the subject.

20. The method of claim 19, wherein the subject has or is suspected of having West syndrome and the method further comprises treating West syndrome in the subject.


SEQUENCE LISTING (21 sequences)

SEQ ID NO: 1 - DNA, 168 bp, synthetic construct
  source 1..168: mol_type = other DNA
gggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatct

SEQ ID NO: 2 - DNA, 145 bp, synthetic construct
  source 1..145: mol_type = other DNA
aggaacccctagtgatggagttggccactccctctctgcgcgctcgctcgctcactgaggccgccggcccaaagggccggcgtcgggcgacctttggtcgcccggcctcagtgagcgagcgagcgcgcagagagggagtggccaa

SEQ ID NO: 3 - DNA, 448 bp, synthetic construct
  source 1..448: mol_type = other DNA
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 4 - DNA, 1480 bp, synthetic construct
  source 1..1480: mol_type = other DNA
gtcgaccgccaccatgctggacatgggagataggaaagaggtgaaaatgatccccaagtcctcgttcagcatcaacagcctggtgcccgaggcggtccagaacgacaaccaccacgcgagccacggccaccacaacagccaccacccccagcaccaccaccaccaccaccaccatcaccaccacccgccgccgcccgccccgcaaccgccgccgccgccgcagcagcagcagccgccgccgccgccgcccccggcaccgcagcccccccagacgcggggcgccccggccgccgacgacgacaagggcccccagcagctgctgctcccgccgccgccaccgccaccaccggccgccgccctggacggggctaaagcggacgggctgggcggcaagggcgagccgggcggcgggccgggggagctggcgcccgtcgggccggacgagaaggagaagggcgccggcgccgggggggaggagaagaagggggcgggcgagggcggcaaggacggggaggggggcaaggagggcgagaagaagaacggcaagtacgagaagccgccgttcagctacaacgcgctcatcatgatggccatccggcagagccccgagaagcggctcacgctcaacggcatctacgagttcatcatgaagaacttcccttactaccgcgagaacaagcagggctggcagaactccatccgccacaatctgtccctcaacaagtgcttcgtgaaggtgccgcgccactacgacgacccgggcaagggcaactactggatgctggacccgtcgagcgacgacgtgttcatcggcggcaccacgggcaagctgcggcgccgctccaccacctcgcgggccaagctggccttcaagcgcggtgcgcgcctcacctccaccggcctcaccttcatggaccgcgccggctccctctactggcccatgtcgcccttcctgtccctgcaccacccccgcgccagcagcactttgagttacaacggcaccacgtcggcctaccccagccaccccatgccctacagctccgtgttgactcagaactcgctgggcaacaaccactccttctccaccgccaacggcctgagcgtggaccggctggtcaacggggagatcccgtacgccacgcaccacctcacggccgccgcgctagccgcctcggtgccctgcggcctgtcggtgccctgctctgggacctactccctcaacccctgctccgtcaacctgctcgcgggccagaccagttactttttcccccacgtcccgcacccgtcaatgacttcgcagagcagcacgtccatgagcgccagggccgcgtcctcctccacgtcgccgcaggccccctcgaccctgccctgtgagtctttaagaccctctttgccaagttttacgacgggactgtctgggggactgtctgattatttcacacatcaaaatcaggggtcttcttccaaccctttaatacat

SEQ ID NO: 5 - protein, 489 aa, synthetic construct [sequence omitted]

SEQ ID NO: 6 - DNA, 30 bp, synthetic construct
  source 1..30: mol_type = other DNA
gaacaaaaactcatctcagaagaggatctg

SEQ ID NO: 7 - protein, 10 aa, synthetic construct
EQKLISEEDL

SEQ ID NO: 8 - DNA, 51 bp, synthetic construct
  source 1..51: mol_type = other DNA
gggagctccggcagttctggagaacaaaaactcatctcagaagaggatctg

SEQ ID NO: 9 - protein, 7 aa, synthetic construct
GSSGSSG

SEQ ID NO: 10 - skipped

SEQ ID NO: 11 - DNA, 175 bp, synthetic construct
  source 1..175: mol_type = other DNA
ctgttctagcccccaaagagttttctgtgcttgcttttgaaacttgaagtcttgaaaaccaaagacatagatgtgaaaattttaggcagtgtaagctgatagcacaagttctggcgactcacaattatgctgtgaattttacaaaaagaagcagtaatctacctcagccgataac

SEQ ID NO: 12 - DNA, 100 bp, synthetic construct
  source 1..100: mol_type = other DNA
caaacaccattgtcacactccaagaccaaacaccattgtcacactccaattacaaacaccattgtcacactccaagaccaaacaccattgtcacactcca

SEQ ID NO: 13 - DNA, 48 bp, synthetic construct
  source 1..48: mol_type = other DNA
aataaagagctcagatgcatcgatcagagtgtgttggttttttgtgtg

SEQ ID NO: 14 - DNA, 168 bp, synthetic construct
  source 1..168: mol_type = other DNA
agatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaaccccccccccccccccc

SEQ ID NO: 15 - DNA, 145 bp, synthetic construct
  source 1..145: mol_type = other DNA
aggaacccctagtgatggagttggccactccctctctgcgcgctcgctcgctcactgaggccgggcgaccaaaggtcgcccgacgccggccctttgggccggcggcctcagtgagcgagcgagcgcgcagagagggagtggccaa

SEQ ID NO: 16 - DNA, 2458 bp, synthetic construct
  source 1..2458: mol_type = other DNA
gggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggtcgaccgccaccatgctggacatgggagataggaaagaggtgaaaatgatccccaagtcctcgttcagcatcaacagcctggtgcccgaggcggtccagaacgacaaccaccacgcgagccacggccaccacaacagccaccacccccagcaccaccaccaccaccaccaccatcaccaccacccgccgccgcccgccccgcaaccgccgccgccgccgcagcagcagcagccgccgccgccgccgcccccggcaccgcagcccccccagacgcggggcgccccggccgccgacgacgacaagggcccccagcagctgctgctcccgccgccgccaccgccaccaccggccgccgccctggacggggctaaagcggacgggctgggcggcaagggcgagccgggcggcgggccgggggagctggcgcccgtcgggccggacgagaaggagaagggcgccggcgccgggggggaggagaagaagggggcgggcgagggcggcaaggacggggaggggggcaaggagggcgagaagaagaacggcaagtacgagaagccgccgttcagctacaacgcgctcatcatgatggccatccggcagagccccgagaagcggctcacgctcaacggcatctacgagttcatcatgaagaacttcccttactaccgcgagaacaagcagggctggcagaactccatccgccacaatctgtccctcaacaagtgcttcgtgaaggtgccgcgccactacgacgacccgggcaagggcaactactggatgctggacccgtcgagcgacgacgtgttcatcggcggcaccacgggcaagctgcggcgccgctccaccacctcgcgggccaagctggccttcaagcgcggtgcgcgcctcacctccaccggcctcaccttcatggaccgcgccggctccctctactggcccatgtcgcccttcctgtccctgcaccacccccgcgccagcagcactttgagttacaacggcaccacgtcggcctaccccagccaccccatgccctacagctccgtgttgactcagaactcgctgggcaacaaccactccttctccaccgccaacggcctgagcgtggaccggctggtcaacggggagatcccgtacgccacgcaccacctcacggccgccgcgctagccgcctcggtgccctgcggcctgtcggtgccctgctctgggacctactccctcaacccctgctccgtcaacctgctcgcgggccagaccagttactttttcccccacgtcccgcacccgtcaatgacttcgcagagcagcacgtccatgagcgccagggccgcgtcctcctccacgtcgccgcaggccccctcgaccctgccctgtgagtctttaagaccctctttgccaagttttacgacgggactgtctgggggactgtctgattatttcacacatcaaaatcaggggtcttcttccaaccctttaatacattaaaggcctctcgagcaaacaccattgtcacactccaagaccaaacaccattgtcacactccaattacaaacaccattgtcacactccaagaccaaacaccattgtcacactccagatatcaataaagagctcagatgcatcgatcagagtgtgttggttttttgtgtgacgcgtgcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaaccccccccccccccccc

SEQ ID NO: 17 - DNA, 2509 bp, synthetic construct
  source 1..2509: mol_type = other DNA
gggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggtcgaccgccaccatgctggacatgggagataggaaagaggtgaaaatgatccccaagtcctcgttcagcatcaacagcctggtgcccgaggcggtccagaacgacaaccaccacgcgagccacggccaccacaacagccaccacccccagcaccaccaccaccaccaccaccatcaccaccacccgccgccgcccgccccgcaaccgccgccgccgccgcagcagcagcagccgccgccgccgccgcccccggcaccgcagcccccccagacgcggggcgccccggccgccgacgacgacaagggcccccagcagctgctgctcccgccgccgccaccgccaccaccggccgccgccctggacggggctaaagcggacgggctgggcggcaagggcgagccgggcggcgggccgggggagctggcgcccgtcgggccggacgagaaggagaagggcgccggcgccgggggggaggagaagaagggggcgggcgagggcggcaaggacggggaggggggcaaggagggcgagaagaagaacggcaagtacgagaagccgccgttcagctacaacgcgctcatcatgatggccatccggcagagccccgagaagcggctcacgctcaacggcatctacgagttcatcatgaagaacttcccttactaccgcgagaacaagcagggctggcagaactccatccgccacaatctgtccctcaacaagtgcttcgtgaaggtgccgcgccactacgacgacccgggcaagggcaactactggatgctggacccgtcgagcgacgacgtgttcatcggcggcaccacgggcaagctgcggcgccgctccaccacctcgcgggccaagctggccttcaagcgcggtgcgcgcctcacctccaccggcctcaccttcatggaccgcgccggctccctctactggcccatgtcgcccttcctgtccctgcaccacccccgcgccagcagcactttgagttacaacggcaccacgtcggcctaccccagccaccccatgccctacagctccgtgttgactcagaactcgctgggcaacaaccactccttctccaccgccaacggcctgagcgtggaccggctggtcaacggggagatcccgtacgccacgcaccacctcacggccgccgcgctagccgcctcggtgccctgcggcctgtcggtgccctgctctgggacctactccctcaacccctgctccgtcaacctgctcgcgggccagaccagttactttttcccccacgtcccgcacccgtcaatgacttcgcagagcagcacgtccatgagcgccagggccgcgtcctcctccacgtcgccgcaggccccctcgaccctgccctgtgagtctttaagaccctctttgccaagttttacgacgggactgtctgggggactgtctgattatttcacacatcaaaatcaggggtcttcttccaaccctttaatacatgggagctccggcagttctggagaacaaaaactcatctcagaagaggatctgtaaaggcctctcgagcaaacaccattgtcacactccaagaccaaacaccattgtcacactccaattacaaacaccattgtcacactccaagaccaaacaccattgtcacactccagatatcaataaagagctcagatgcatcgatcagagtgtgttggttttttgtgtgacgcgtgcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaaccccccccccccccccc

SEQ ID NO: 18 - DNA, 2684 bp, synthetic construct
  source 1..2684: mol_type = other DNA
gggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggtcgaccgccaccatgctggacatgggagataggaaagaggtgaaaatgatccccaagtcctcgttcagcatcaacagcctggtgcccgaggcggtccagaacgacaaccaccacgcgagccacggccaccacaacagccaccacccccagcaccaccaccaccaccaccaccatcaccaccacccgccgccgcccgccccgcaaccgccgccgccgccgcagcagcagcagccgccgccgccgccgcccccggcaccgcagcccccccagacgcggggcgccccggccgccgacgacgacaagggcccccagcagctgctgctcccgccgccgccaccgccaccaccggccgccgccctggacggggctaaagcggacgggctgggcggcaagggcgagccgggcggcgggccgggggagctggcgcccgtcgggccggacgagaaggagaagggcgccggcgccgggggggaggagaagaagggggcgggcgagggcggcaaggacggggaggggggcaaggagggcgagaagaagaacggcaagtacgagaagccgccgttcagctacaacgcgctcatcatgatggccatccggcagagccccgagaagcggctcacgctcaacggcatctacgagttcatcatgaagaacttcccttactaccgcgagaacaagcagggctggcagaactccatccgccacaatctgtccctcaacaagtgcttcgtgaaggtgccgcgccactacgacgacccgggcaagggcaactactggatgctggacccgtcgagcgacgacgtgttcatcggcggcaccacgggcaagctgcggcgccgctccaccacctcgcgggccaagctggccttcaagcgcggtgcgcgcctcacctccaccggcctcaccttcatggaccgcgccggctccctctactggcccatgtcgcccttcctgtccctgcaccacccccgcgccagcagcactttgagttacaacggcaccacgtcggcctaccccagccaccccatgccctacagctccgtgttgactcagaactcgctgggcaacaaccactccttctccaccgccaacggcctgagcgtggaccggctggtcaacggggagatcccgtacgccacgcaccacctcacggccgccgcgctagccgcctcggtgccctgcggcctgtcggtgccctgctctgggacctactccctcaacccctgctccgtcaacctgctcgcgggccagaccagttactttttcccccacgtcccgcacccgtcaatgacttcgcagagcagcacgtccatgagcgccagggccgcgtcctcctccacgtcgccgcaggccccctcgaccctgccctgtgagtctttaagaccctctttgccaagttttacgacgggactgtctgggggactgtctgattatttcacacatcaaaatcaggggtcttcttccaaccctttaatacatgggagctccggcagttctggagaacaaaaactcatctcagaagaggatctgtaaaggcctctgttctagcccccaaagagttttctgtgcttgcttttgaaacttgaagtcttgaaaaccaaagacatagatgtgaaaattttaggcagtgtaagctgatagcacaagttctggcgactcacaattatgctgtgaattttacaaaaagaagcagtaatctacctcagccgataacctcgagcaaacaccattgtcacactccaagaccaaacaccattgtcacactccaattacaaacaccattgtcacactccaagaccaaacaccattgtcacactccagatatcaataaagagctcagatgcatcgatcagagtgtgttggttttttgtgtgacgcgtgcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaaccccccccccccccccc

SEQ ID NO: 19 - DNA, 2633 bp, synthetic construct
  source 1..2633: mol_type = other DNA
gggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggtcgaccgccaccatgctggacatgggagataggaaagaggtgaaaatgatccccaagtcctcgttcagcatcaacagcctggtgcccgaggcggtccagaacgacaaccaccacgcgagccacggccaccacaacagccaccacccccagcaccaccaccaccaccaccaccatcaccaccacccgccgccgcccgccccgcaaccgccgccgccgccgcagcagcagcagccgccgccgccgccgcccccggcaccgcagcccccccagacgcggggcgccccggccgccgacgacgacaagggcccccagcagctgctgctcccgccgccgccaccgccaccaccggccgccgccctggacggggctaaagcggacgggctgggcggcaagggcgagccgggcggcgggccgggggagctggcgcccgtcgggccggacgagaaggagaagggcgccggcgccgggggggaggagaagaagggggcgggcgagggcggcaaggacggggaggggggcaaggagggcgagaagaagaacggcaagtacgagaagccgccgttcagctacaacgcgctcatcatgatggccatccggcagagccccgagaagcggctcacgctcaacggcatctacgagttcatcatgaagaacttcccttactaccgcgagaacaagcagggctggcagaactccatccgccacaatctgtccctcaacaagtgcttcgtgaaggtgccgcgccactacgacgacccgggcaagggcaactactggatgctggacccgtcgagcgacgacgtgttcatcggcggcaccacgggcaagctgcggcgccgctccaccacctcgcgggccaagctggccttcaagcgcggtgcgcgcctcacctccaccggcctcaccttcatggaccgcgccggctccctctactggcccatgtcgcccttcctgtccctgcaccacccccgcgccagcagcactttgagttacaacggcaccacgtcggcctaccccagccaccccatgccctacagctccgtgttgactcagaactcgctgggcaacaaccactccttctccaccgccaacggcctgagcgtggaccggctggtcaacggggagatcccgtacgccacgcaccacctcacggccgccgcgctagccgcctcggtgccctgcggcctgtcggtgccctgctctgggacctactccctcaacccctgctccgtcaacctgctcgcgggccagaccagttactttttcccccacgtcccgcacccgtcaatgacttcgcagagcagcacgtccatgagcgccagggccgcgtcctcctccacgtcgccgcaggccccctcgaccctgccctgtgagtctttaagaccctctttgccaagttttacgacgggactgtctgggggactgtctgattatttcacacatcaaaatcaggggtcttcttccaaccctttaatacattaaaggcctctgttctagcccccaaagagttttctgtgcttgcttttgaaacttgaagtcttgaaaaccaaagacatagatgtgaaaattttaggcagtgtaagctgatagcacaagttctggcgactcacaattatgctgtgaattttacaaaaagaagcagtaatctacctcagccgataacctcgagcaaacaccattgtcacactccaagaccaaacaccattgtcacactccaattacaaacaccattgtcacactccaagaccaaacaccattgtcacactccagatatcaataaagagctcagatgcatcgatcagagtgtgttggttttttgtgtgacgcgtgcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaaccccccccccccccccc

SEQ ID NO: 20 - DNA, 1467 bp, Homo sapiens
  source 1..1467: mol_type = other DNA
atgctggacatgggagataggaaagaggtgaaaatgatccccaagtcctcgttcagcatcaacagcctggtgcccgaggcggtccagaacgacaaccaccacgcgagccacggccaccacaacagccaccacccccagcaccaccaccaccaccaccaccatcaccaccacccgccgccgcccgccccgcaaccgccgccgccgccgcagcagcagcagccgccgccgccgccgcccccggcaccgcagcccccccagacgcggggcgccccggccgccgacgacgacaagggcccccagcagctgctgctcccgccgccgccaccgccaccaccggccgccgccctggacggggctaaagcggacgggctgggcggcaagggcgagccgggcggcgggccgggggagctggcgcccgtcgggccggacgagaaggagaagggcgccggcgccgggggggaggagaagaagggggcgggcgagggcggcaaggacggggaggggggcaaggagggcgagaagaagaacggcaagtacgagaagccgccgttcagctacaacgcgctcatcatgatggccatccggcagagccccgagaagcggctcacgctcaacggcatctacgagttcatcatgaagaacttcccttactaccgcgagaacaagcagggctggcagaactccatccgccacaatctgtccctcaacaagtgcttcgtgaaggtgccgcgccactacgacgacccgggcaagggcaactactggatgctggacccgtcgagcgacgacgtgttcatcggcggcaccacgggcaagctgcggcgccgctccaccacctcgcgggccaagctggccttcaagcgcggtgcgcgcctcacctccaccggcctcaccttcatggaccgcgccggctccctctactggcccatgtcgcccttcctgtccctgcaccacccccgcgccagcagcactttgagttacaacggcaccacgtcggcctaccccagccaccccatgccctacagctccgtgttgactcagaactcgctgggcaacaaccactccttctccaccgccaacggcctgagcgtggaccggctggtcaacggggagatcccgtacgccacgcaccacctcacggccgccgcgctagccgcctcggtgccctgcggcctgtcggtgccctgctctgggacctactccctcaacccctgctccgtcaacctgctcgcgggccagaccagttactttttcccccacgtcccgcacccgtcaatgacttcgcagagcagcacgtccatgagcgccagggccgcgtcctcctccacgtcgccgcaggccccctcgaccctgccctgtgagtctttaagaccctctttgccaagttttacgacgggactgtctgggggactgtctgattatttcacacatcaaaatcaggggtcttcttccaaccctttaatacat

SEQ ID NO: 21 - DNA, 13 bp, synthetic construct
  source 1..13: mol_type = other DNA
gtcgaccgccacc